(19) United States
(12) Patent Application Publication
(10) Pub. No.: US 2023/0380692 A1
(43) Pub. Date: Nov. 30, 2023

(54) OPTIMIZED STRUCTURES FOR OPTICAL MEASUREMENT

(71) Applicant: Oura Health Oy, Oulu (FI)

(72) Inventors: Heikki Juhani Huttunen, Haukipudas (FI); Jukka-Tapani Mäkinen, Oulu (FI); Antti Lämsä, Oulu (FI); Antti Saikkonen, Haukipudas (FI); Kari Kanniainen, Ii (FI); Marko Uusitalo, Oulu (FI); Marko Kelloniemi, Oulu (FI); Sami Ihme, Oulu (FI); Tapani Vaskuri, Revonlahti (FI); Teemu Haverinen, Oulu (FI)

(21) Appl. No.: 17/826,368

(22) Filed: May 27, 2022

(51) Int. Cl. A61B 5/00; G06F 1/16

(52) U.S. Cl. CPC A61B 5/0064; G06F 1/163; A61B 5/6826; A61B 5/6802

(57) ABSTRACT

Systems and devices for optimized structures for optical measurement are described. A wearable device may include a housing configured to house one or more sensors to acquire physiological data from a user. The wearable device may further include one or more light sources disposed on a surface of the housing and positioned to direct light into a tissue surface of the user and one or more detectors disposed on the surface of the housing and positioned to receive light from the one or more light sources along a plurality of optical paths where a first optical path may be through the tissue surface and a second optical path may be directly from the one or more light sources. The wearable device may include one or more light blocking components disposed on a surface of the housing that are configured to block stray light along the second optical path.

### FIELD OF TECHNOLOGY

The following relates to wearable devices and data processing, including optimized structures for optical measurement.

### BACKGROUND

Some wearable devices may be configured to collect data from users, including temperature data, heart rate data, and the like. However, light that travels directly between a light source and a detector of the wearable device without traveling through a user's skin may result in inaccurate measurements.

### BRIEF DESCRIPTION OF THE DRAWINGS

FIG. 1 illustrates an example of a system that supports optimized structures for optical measurement in accordance with aspects of the present disclosure.

FIG. 2 illustrates an example of a system that supports optimized structures for optical measurement in accordance with aspects of the present disclosure.

FIG. 3 illustrates an example of a wearable device diagram that supports optimized structures for optical measurement in accordance with aspects of the present disclosure.

FIG. 4 illustrates an example of a wearable device diagram that supports optimized structures for optical measurement in accordance with aspects of the present disclosure.

FIG. 5A illustrates an example of a perspective view of a wearable device that supports optimized structures for optical measurement in accordance with aspects of the present disclosure.

FIG. 5B illustrates an example of a cross-sectional view of the wearable device illustrated in FIG. 5A that supports optimized structures for optical measurement in accordance with aspects of the present disclosure.

FIG. 6A illustrates an example of a perspective view of a wearable device that supports optimized structures for optical measurement in accordance with aspects of the present disclosure.

FIG. 6B illustrates an example of a cross-sectional view of the wearable device illustrated in FIG. 6A that supports optimized structures for optical measurement in accordance with aspects of the present disclosure.

FIG. 7A illustrates an example of a perspective view of a wearable device that supports optimized structures for optical measurement in accordance with aspects of the present disclosure.

FIG. 7B illustrates an example of a cross-sectional view of the wearable device illustrated in FIG. 7A that supports optimized structures for optical measurement in accordance with aspects of the present disclosure.

FIG. 8A illustrates an example of a perspective view of a wearable device that supports optimized structures for optical measurement in accordance with aspects of the present disclosure.

FIG. 8B illustrates an example of a cross-sectional view of the wearable device illustrated in FIG. 8A that supports optimized structures for optical measurement in accordance with aspects of the present disclosure.

FIG. 9A illustrates an example of a perspective view of a wearable device that supports optimized structures for optical measurement in accordance with aspects of the present disclosure.

FIG. 9B illustrates an example of a cross-sectional view of the wearable device illustrated in FIG. 9A that supports optimized structures for optical measurement in accordance with aspects of the present disclosure.

FIG. 10A illustrates an example of a perspective view of a wearable device that supports optimized structures for optical measurement in accordance with aspects of the present disclosure.

FIG. 10B illustrates an example of a cross-sectional view of the wearable device illustrated in FIG. 10A that supports optimized structures for optical measurement in accordance with aspects of the present disclosure.

### DETAILED DESCRIPTION

Some wearable devices may be configured to collect data from users associated with movement and other activities. For example, some wearable devices may be configured to continuously acquire physiological data associated with a user including temperature data, heart rate data, and the like. As such, some wearable devices may be configured to house one or more sensors configured to acquire physiological data from a user. In some cases, a wearable device may include a flexible printed circuit board (PCB) including electrical circuitry for the one or more sensors. The wearable device may include one or more light sources (e.g., light emitting diodes (LEDs) or other types of light sources) positioned to direct light into a tissue surface of the user and one or more detectors (e.g., photodetectors) positioned to receive the light that passes at least partially through the tissue surface.

In some cases, the wearable devices may include a layer of material disposed on the surface of a housing where the material includes optical properties that propagates light within the layer of the material. For example, the layer of material may be an example of an inlay of the housing where the layer of material may be any type of material capable of transmitting light through it. The one or more detectors may receive light directly from the one or more light sources that propagates within the layer of material. As such, the one or more detectors may receive stray light that passes through the layer of material without passing at least partially through the tissue surface, thereby introducing and increasing an amount of noise in the signal associated with the acquired physiological data (e.g., a photoplethysmogram (PPG) signal) and decreasing the efficiency and accuracy of the signal.

Moreover, a user's movement may displace or damage components of some wearable devices, which may detrimentally affect the ability of the wearable device to efficiently and accurately acquire physiological data and increase an amount of the noise in the signal due to the light dispersion. The presence of internal stray light (e.g., within the layer of material) may reduce the perfusion index (e.g., ratio of the pulsatile blood flow to the static blood flow) and cause inaccurate readings from the sensors. Taken together, these issues with wearable devices may result in inaccurate physiological data readings, which may lead to a distorted picture of the user's overall health, as well as increased power consumption and decreased battery life. As such, conventional techniques for obtaining optical measurements within the device are deficient for multiple reasons.

Accordingly, to facilitate improved health monitoring, aspects of the present disclosure are directed to optimized structures within the wearable device for optical measurement. For example, the wearable device may include one or more light blocking components disposed on the surface of the housing. The one or more light blocking components may be configured to allow light from the one or more light sources that passes at least partially through the tissue surface to enter the one or more detectors. For example, the one or more light blocking components may be configured to allow light to enter the one or more detectors from the one or more light sources along a first optical path that passes at least partially through the tissue surface.

The one or more light blocking components may be configured to block light directly from the one or more light sources within the layer of material from entering the one or more detectors. For example, the one or more light blocking components may be configured to block light from the one or more light sources along a second optical path directly from one or more of the one or more light sources to one or more of the one or more detectors. The light blocking components may be an example of one or more grooves embedded within the layer of the material, one or more opaque structures attached to the housing around the one or more detectors, one or more opaque structures attached to the housing around the one or more light sources, an increased height of the one or more detectors, an angular filter adhered to the one or more detectors, or a combination thereof.

In such cases, one or more light blocking components may help limit the field of view of the one or more detectors, the one or more light sources, or both to be towards the tissue surface and along the first optical path. For example, the one or more light blocking components may limit the field of view of the one or more detectors to be away from the one or more light sources along the second optical path within the material, thereby decreasing an amount of noise in the signal and increasing the efficiency and accuracy of the signal. By implementing one or more light blocking features on the surface of the housing within the wearable device, techniques described herein may reduce an amount of internal stray light within the material and may lead to more accurate physiological data measurements.

Aspects of the disclosure are initially described in the context of systems supporting physiological data collection from users via wearable devices. Additional aspects of the disclosure are described in the context of example rings. Although many of the examples of a wearable device depicted herein are ring-shaped wearable devices, it should be understood that the light blocking features described herein may also be used in wearable devices of other form factors such as watches, patches, and the like.

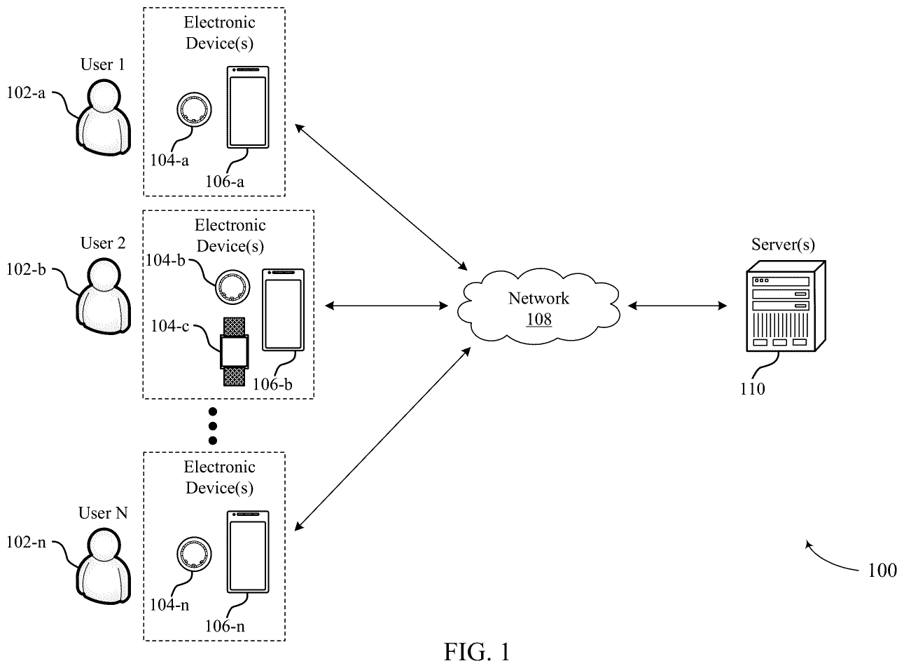

FIG. 1 illustrates an example of a system 100 that supports optimized structures for optical measurement in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user's 102 finger, a wrist computing device (e.g., a smart watch, fitness band, or bracelet) configured to be worn on a user's 102 wrist, and/or a head mounted computing device (e.g., glasses/goggles). Wearable devices 104 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing, or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, heart rate variability (HRV), actigraphy, galvanic skin response, pulse oximetry, and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled to one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled to the user devices 106 of the respective users 102 via Bluetooth, Wi-Fi, and other wireless protocols.

In some implementations, the rings 104 (e.g., wearable devices 104) of the system 100 may be configured to collect physiological data from the respective users 102 based on arterial blood flow within the user's finger. In particular, a ring 104 may utilize one or more LEDs (e.g., red LEDs, green LEDs) that emit light on the palm-side of a user's finger to collect physiological data based on arterial blood flow within the user's finger. In some cases, the system 100 may be configured to collect physiological data from the respective users 102 based on blood flow diffused into a microvascular bed of skin with capillaries and arterioles. For example, the system 100 may collect PPG data based on a measured amount of blood diffused into the microvascular system of capillaries and arterioles. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the system 100 may detect periods of time during which a user 102 is asleep, and classify periods of time during which the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects, data collected by the ring 104-a may be input to a machine learning classifier, where the machine learning classifier is configured to determine periods of time during which the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 102-a via a GUI of the user device 106-a. Sleep stage classification may be used to provide feedback to a user 102-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 100 may utilize circadian rhythm-derived features to further improve physiological data collection, data processing procedures, and other techniques described herein. The term circadian rhythm may refer to a natural, internal process that regulates an individual's sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model, and may modify the baseline model using physiological data collected from each user 102 to generate tailored, individualized circadian rhythm adjustment models that are specific to each respective user 102.

In some aspects, the system 100 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g. in a hypothetical culture with 12 day “weeks”, 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

In some aspects, the respective devices of the system 100 may support an apparatus for a wearable device 104 including one or more light blocking components of the wearable device 104. For example, a wearable device 104 may include a housing configured to house one or more sensors configured to acquire physiological data from a user 102. The wearable device 104 may include one or more light sources and one or more detectors disposed on a surface of the housing. The one or more light sources may be positioned to direct light into a tissue surface of the user 102 and the one or more detectors may be positioned to receive light from the one or more light sources along one or more of a plurality of optical paths.

In some implementations, the one or more detectors may be positioned to receive light from a first optical path of the plurality of optical paths that is through (e.g., passes at least partially through) the tissue surface, to receive light from a second optical path of the plurality of optical paths that passes directly from the one or more light sources to the one or more detectors, or both. The wearable device 104 may include one or more light blocking components disposed on the surface of the housing. The one or more light blocking components are configured to block light from the one or more light sources along the second optical path directly from the one or more light sources. The one or more light blocking components may be configured to allow light from the one or more light sources along the first optical path to enter the one or more detectors.

The wearable device 104 may surround a finger, wrist, ankle, or the like, of a user 102. The wearable device 104 may take measurements via the one or more sensors (e.g., heart rate measurements, oxygen saturation measurements (SpO2), temperature, sleep measurements, and the like). In some cases, the wearable device 104 may be displaced by a force, may shake due to external forces or gravity, may rotate, or may move in some other way to cause changes in skin contact between the tissue surface of a user and one or more sensors on the wearable device. In such cases, a signal associated with the physiological data acquired by the one or more sensors may experience noise and result in inaccurate measurements.

In some cases, the wearable device 104 may propagate light (e.g., stray light) along the second optical path directly from the one or more light sources to the one or more detectors. In such cases, the one or more detectors may receive light directly from the one or more light sources rather than through the tissue surface. For example, the signal associated with the physiological data acquired by the one or more sensors may experience noise and result in inaccurate measurements due to the stray light that travels directly from the one or more light sources without traveling through the tissue surface. In such cases, the wearable device 104 may include one or more light blocking components to block the stray light along the second optical path, thereby improving the signal quality strength, and accuracy of measurements, as described herein.

It should be appreciated by a person skilled in the art that one or more aspects of the disclosure may be implemented in a system 100 to additionally or alternatively solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

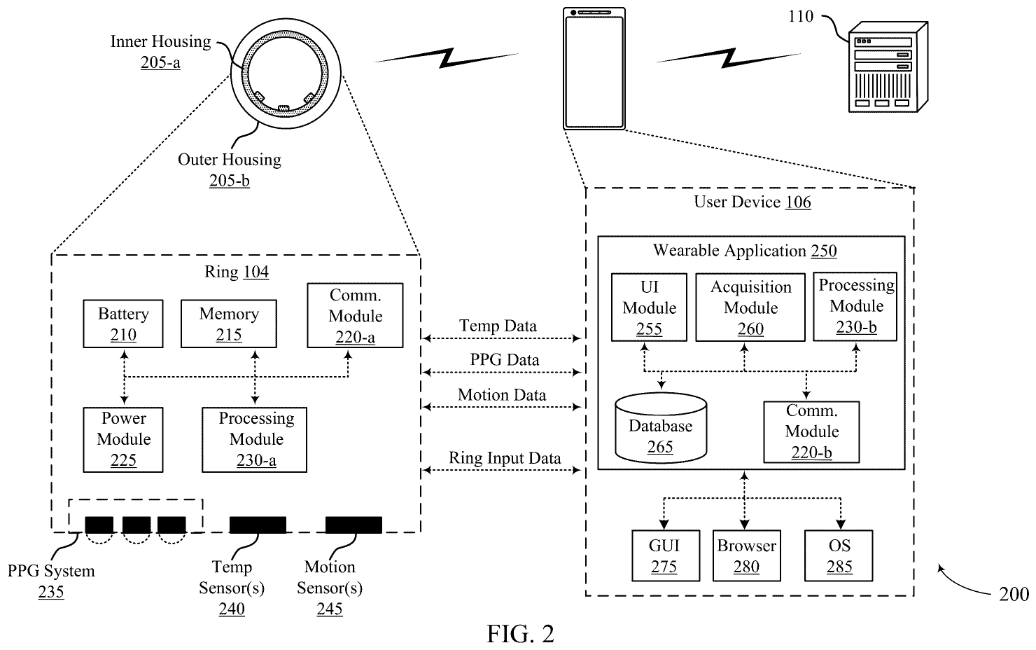

FIG. 2 illustrates an example of a system 200 that supports optimized structures for optical measurement in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects, the ring 104 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels, and the like.

The system 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, photoplethysmogram (PPG) data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205 that may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled to one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user's finger (e.g., using a clamps, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, that may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user's finger. The inner housing 205-a may be formed from a polymer (e.g., a medical grade polymer) or other material. In some implementations, the inner housing 205-a may be transparent. For example, the inner housing 205-a may be transparent to light emitted by the PPG light emitting diodes (LEDs). In some implementations, the inner housing 205-a component may be molded onto the outer housing 205-b. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user's finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors that may be used to collect data in addition to, or which supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during 104 charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during 104 charging, and under voltage during 104 discharge. The power module 225 may also include electro-static discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user's finger (e.g., skin temperature). In some implementations, the temperature sensor 240 may contact the user's skin. In other implementations, a portion of the housing 205 (e.g., the inner housing 205-a) may form a barrier (e.g., a thin, thermally conductive barrier) between the temperature sensor 240 and the user's skin. In some implementations, portions of the ring 104 configured to contact the user's finger may have thermally conductive portions and thermally insulative portions. The thermally conductive portions may conduct heat from the user's finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user's temperature over time. For example, the processing module 230-a may sample the user's temperature according to a sampling rate. An example sampling rate may include one sample per second, although the processing module 230-a may be configured to sample the temperature signal at other sampling rates that are higher or lower than one sample per second. In some implementations, the processing module 230-a may sample the user's temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, which may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during 104 exercise (e.g., as indicated by a motion sensor 245).

The ring 104 (e.g., communication module) may transmit the sampled and/or average temperature data to the user device 106 for storage and/or further processing.

The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user's finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user's finger (e.g., any finger). For example, one or more temperature sensors 240 on the ring 104 may acquire a user's temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 235 may indicate the perfusion of blood in the illuminated region. For example, the PPG signal may indicate blood volume changes in the illuminated region caused by a user's pulse pressure. The processing module 230-a may sample the PPG signal and determine a user's pulse waveform based on the PPG signal. The processing module 230-a may determine a variety of physiological parameters based on the user's pulse waveform, such as a user's respiratory rate, heart rate, HRV, oxygen saturation, and other circulatory parameters.

In some implementations, the PPG system 235 may be configured as a reflective PPG system 235 in which the optical receiver(s) receive transmitted light that is reflected through the region of the user's finger. In some implementations, the PPG system 235 may be configured as a transmissive PPG system 235 in which the optical transmitter(s) and optical receiver(s) are arranged opposite to one another, such that light is transmitted directly through a portion of the user's finger to the optical receiver(s).

The number and ratio of transmitters and receivers included in the PPG system 235 may vary. Example optical transmitters may include light-emitting diodes (LEDs). The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user's heart rate based on the pulse waveform. For example, the processing module 230-a may determine heart rate (e.g., in beats per minute) based on the time between peaks in the pulse waveform. The time between peaks may be referred to as an interbeat interval (IBI). The processing module 230-a may store the determined heart rate values and IBI values in memory 215.

The processing module 230-a may determine HRV over time. For example, the processing module 230-a may determine HRV based on the variation in the IBls. The processing module 230-a may store the HRV values over time in the memory 215. Moreover, the processing module 230-a may determine the user's respiratory rate over time. For example, the processing module 230-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BM1160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user's finger and if the ring 104 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user's physiological parameters may be measured by sensors included on a ring 104, other devices may measure a user's physiological parameters. For example, although a user's temperature may be measured by a temperature sensor 240 included in a ring 104, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during 104 portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS), a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that require relatively low processing power and/or operations that require a relatively low latency, whereas the user device 106 may transmit the data to the servers 110 for processing operations that require relatively high processing power and/or operations that may allow relatively higher latency.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time in which the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5° C. higher than the highest temperature during the longest period. In some aspects, the ring may measure a user's body temperature while the user is asleep, and the system 200 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

In some aspects, the system 200 may support a wearable device 104 including optimized structures for optical measurement from one or more sensors of the wearable device 104. In particular, techniques described herein support a wearable device 104, such as a wearable device 104 as described with reference to FIG. 1. For example, a wearable device 104 may include an inner housing 205-a configured to house one or more sensors configured to acquire physiological data from a user 102 and an outer housing 205-b configured to house the inner housing 205-a. As described in more detail herein, the inner housing 205-a may be made from a material that propagates light (e.g., an epoxy or similar material). The one or more sensors may take physiological measurements from the user (e.g., temperature sensors, additional LED-PD sensors used for measuring heart rate, oxygen saturation, one or more sensors that a device may use to detect whether a user is asleep, or the like). In some cases, the one or more sensors are configured to acquire the physiological data from the user based on arterial blood flow. In some implementations, the one or more sensors are configured to acquire the physiological data (e.g., including PPG data) from the user based on blood flow that is diffused into the microvascular bed of skin with capillaries and arterioles. The one or more sensors may be an example of photodetectors from the PPG system 235, temperature sensors 240, motion sensors 245, and other sensors.

The wearable device 104 may include one or more light sources disposed on a surface of the inner housing 205-a and positioned to direct light into a tissue surface of the user 102. The wearable device 104 may include one or more detectors disposed on the surface of the inner housing 205-a and positioned to receive light from the one or more light sources along a plurality of optical paths. For example, a first optical path of the plurality of optical paths passes at least partially through the tissue surface, and a second optical path of the plurality of optical paths passes directly from the one or more light source to the one or more detectors (e.g., without first at least partially passing through the tissue surface). In some implementations, the wearable device may include one or more light blocking components disposed on the surface of the inner housing 205-a. In such cases, the one or more light blocking components are configured to block light from the one or more lights sources along the second optical path from entering the one or more detectors. The one or more light blocking components may be configured to allow light from the one or more light sources along the first optical path to enter the one or more detectors.

While much of the present disclosure describes one or more light blocking components in the context of a wearable ring device, aspects of the present disclosure may additionally or alternatively be implemented in the context of other wearable devices. For example, in some implementations, the one or more light blocking components described herein may be implemented in the context of other wearable devices, such as bracelets, watches, necklaces, piercings, and the like. For example, the wearable device 104 may surround a finger, wrist, ankle, or the like of a user.

A plurality of optical paths of the wearable device 104 may direct light from a light source, such as from a colored LED light located at inner housing 205-a, to a detector, which may also be located at inner housing 205-a. The LED light may be a red LED light, an infrared LED light, a blue LED light, a yellow LED light, a green LED light, or some other color LED light. The first optical path may be characterized as light that travels from a light source and at least partially through the tissue surface before being received by a detector. Light that passes through the first optical path may be used by the system (e.g., the PPG system 235) to measure physiological parameters from the blood flow of a user. The second optical path may be characterized as light that travels within the inner housing 205-a and travels directly from the light source to the detector without first entering or passing through a tissue surface of the user. In some cases, the inner housing 205-a may be made from a material that propagates light (e.g., epoxy or the like), thereby providing an opportunity for light to travel along the second optical path. Since light that traveled along the second optical path did not first pass through the tissue surface, this light does not provide measurement information from the blood flow, and thereby may add noise to the overall light signal being received at a light detector. As described herein, one or more physical features (e.g., light blockers or the like), may be arranged within or on the inner housing 205-a to prevent light traveling along the second optical path from entering into a light detector, while still allowing light traveling along the first optical path to enter the light detector.

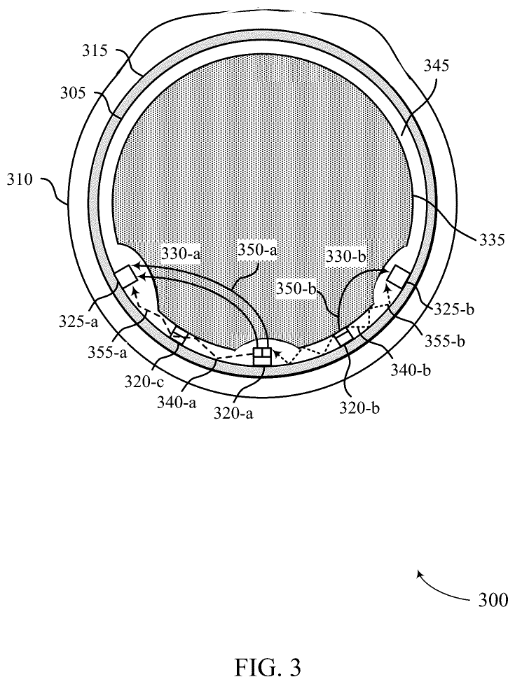

FIG. 3 illustrates an example of a wearable device diagram 300 that supports optimized structures for optical measurement in accordance with aspects of the present disclosure. The wearable device diagram 300 may implement, or be implemented by, aspects of the system 100, system 200, or both. For example, wearable device diagram 300 may illustrate examples of wearable devices 104 as described with reference to FIG. 1. Specifically, the wearable device diagram 300 may illustrate an orientation of a wearable ring device on a user's finger. Although the wearable device is illustrated as a ring in FIG. 3, it may be any example of a wearable device (e.g., a watch, a necklace, and the like).

The wearable device in wearable device diagram 300 may include an inner housing 305 and an outer housing 310, which may be examples of an inner housing 205-a and an outer housing 205-b as described with reference to FIG. 2. In some cases, an outer opaque shell may be molded over an inner structure of the wearable device.

Further, the wearable device in the wearable device diagram 300 may include an electronic substrate 315, such as a printed wiring board (PWB) or PCB. The PWB may have both flexible and rigid sections. One or more sensors may be embedded in the electronic substrate 315. For example, the electronic substrate 315 may include one or more light sources 320 and detectors 325. The light sources 320 may be an example of LED lights may be a blue LED light, a yellow LED light, a green LED light, a red light, an IR light, or some other color LED light. In some cases, the light sources 320 may be an example of a laser diode (LD).

The wearable device may include light source 320-a, which may emit light 330-a received by detector 325-a and/or detector 325-b. In this regard, the light source 320-a may support one or more first optical paths 350 through the tissue 335 for physiological data measurements. For instance, the light source 320-a may support a first optical path 350-a between the light source 320-a and the detector 325-a and another first optical path between the light source 320-a and the detector 325-b. The wearable device may include any number of light sources, detectors, and respective optical paths for physiological data measurements. In some cases, the light source 320-a may be a red and infrared LED, which may emit light 330-a that is scattered and absorbed by the tissue 335 of a user of the wearable device.

Similarly, the wearable device may include light source 320-b and light source 320-c. For example, the light source 320-b may emit light 330-b. The light source 320-b and the light source 320-c may be green LEDs, blue LEDs, or a combination thereof (e.g., one blue LED and one green LED). The light 330-b may be scattered and absorbed by the tissue 335 of the user, and measured via the detectors 325-a and/or 325-b. As noted previously herein, each of the light sources 320-b and 320-c may support one or more first optical paths 350 via the respective detectors 325-a and 325-b. For instance, the light source 320-b may support a first optical path 350-b between the light source 320-b and the detector 325-b and another first optical path between the light source 320-b and the detector 325-a. The light source 320-c may support a first optical path between the light source 320-c and the detector 325-b and another first optical path between the light source 320-c and the detector 325-a. The detectors 325-a and 325-b may be configured to measure light 330 from the respective light sources 320 which is reflected by the tissue 335 and/or transmitted through the tissue 335 (e.g., reflective and/or transmissive measurements). In such cases, the light 330 may be used for physiological data measurements associated with the user.

In some examples, the inner housing 305 may include a dome structure over the one or more light sources 320, one or more detectors 325, or both. For example, the wearable device may include dome structures over the light source 320-a, the detector 325-a, and the detector 325-b to improve contact with the tissue 335. In some other cases, there may be a window for the light source 320 to emit the light 330. For example, the light source 320-b and the light source 320-c may each have a window in the inner housing 305. An optical interface may form between the inner housing 305 and the domes or the windows (e.g., with a refractive index of ˜1.57) and the top layer of the tissue 335 (e.g., with a refractive index of ˜1.55). The wearable device may use the light propagation from the light sources 320 to the detectors 325 through the tissue 335 and along the one or more first optical paths 350 for physiological measurements, such as PPG and SpO2 measurements. That is, the wearable device may use the light 330-a from the light source 320-a, which may include red and infrared wavelengths, to measure SpO2 and the light 330-b from the light source 320-b or light source 320-c, which may include green wavelengths, to measure PPG. The light 330-a may penetrate the tissue 335 to a different depth than the light 330-b.

The wearable device may include a layer of material 345. In some cases, the layer of material 345 may interface with the inner housing 305 and the surface of tissue 335. The layer of material 345 may be disposed on the surface of the housing (e.g., inner housing 305). In some examples, the layer of material 345 may be molded from a material (e.g., epoxy, plastic, and the like) that is capable of transmitting light. That is, the layer of material 345 may have optical properties that allow the layer of material 345 to propagate light from the light sources 320 within the inner housing 305. For example, the layer of material 345 may propagate light 340 (e.g., stray light) along one or more second optical paths 355 within the layer of material 345. For instance, the light source 320-a may support a second optical path 355-a between the light source 320-a and the detector 325-a and another second optical path between the light source 320-a and the detector 325-b. In such cases, the detector 325-a may receive light 340-a along the second optical path 355-a that passes directly from the light source 320-a (e.g., within and/or along the surface of the layer of material 345). Similarly, the light source 320-b may support a second optical path 355-b between the light source 320-b and the detector 325-b and another second optical path between the light source 320-b and the detector 325-a. The light source 320-c may support a second optical path between the light source 320-c and the detector 325-b and another second optical path between the light source 320-c and the detector 325-a. In such cases, the detector 325-b may receive light 340-b along the second optical path 355-b that passes directly from the light source 320-b (e.g., within and/or along the surface of the layer of material 345).

In some cases, stray light emitted directly from the light sources 320 to the detectors 325 (e.g., the light 340 propagating along the second optical paths 355) may impact physiological measurements for the user of the wearable device. For instance, the detector 325-a of the wearable device may detect the light 330-a along the first optical path 350-a through the tissue 335 and the light 340-a along the second optical path 355 directly from the light source 320-a (e.g., not through the tissue 335). The light 330 along the first optical paths 350 may be used for physiological data measurements associated with the user while the light 340 along the second optical paths 355 may impact the accuracy of the physiological measurements of the user of the wearable device.

In some examples, the stray light (e.g., the light 340-a and the light 340-b) signal on a detector may contain an alternating current (AC) component and a direct current (DC) component. The AC component may be an example of a pulsating component of the stray light that may result from penetration of the tissue 335. Accordingly, the stray light may provide information that is relevant to the physiological measurements (e.g., heart rate information). Additionally, the DC component may be an example of a background component of the stray light detected by the detectors 325. Because the light 340 may propagate through a shallow penetration depth of the layer of material 345 along the optical paths 355, the stray light may contain a proportionally larger DC component than AC component. In such cases, the detectors 325 may detect a larger background component than information relevant to the physiological measurements of the user due to the presence of stray light (e.g., light 340) within the layer of material 345.

In some examples, the quality of the physiological measurements may be determined based on a perfusion index. The perfusion index of the signal detected by the detectors 325 (e.g., including the light 330-a and the light 340-a) may be defined as a ratio between the AC component and the DC component of the signal. Accordingly, the perfusion index may decrease if the DC component increases and the AC component is constant. Similarly, the perfusion index may increase if the DC component decreases and the AC component is constant. In some aspects, a lower perfusion index may indicate a lower quality signal for the physiological measurements because a lower perfusion index may indicate that the AC component is proportionally smaller than the DC component, thereby indicating a proportionally smaller amount of information relevant to the physiological measurements than a background component of the signal. The detectors 325 may improve the quality and accuracy of the physiological measurements by detecting light with a higher perfusion index. For instance, SpO2 measurements may be sensitive to stray light due to the larger DC component that is associated with the stray light, although heart rate measurements may be less sensitive to the stray light than the SpO2 measurements because the AC component detected by the detectors 325 may be minimally affected.

In addition to the scattering and absorption properties of the tissue 335, the multiple interfaces between optical features and the tissue 335 may determine how well the optical signal is transmitted from the light sources 320 to the detectors 325. For example, with good skin contact and embedded light sources 320, a relatively influential optical interface type is between the inner housing 305 and the tissue 335, such as the skin outer layer, stratum corneum. The total internal reflection (TIR) critical angle may be relatively large over the optical interface (e.g., 81 degrees), and light out-coupling from the inner housing 305 may be relatively efficient (e.g., <0.1% light lost at the interface via Fresnel reflections). Thus, total light coupling losses from the light sources 320 to the tissue 335 may be relatively low. The TIR is an optical phenomenon when light propagating inside optically clear material hits an interface between the material and another optical material with lower refractive index, the light may be totally reflected back into light source 320 if the angle of incidence is large enough. The TIR critical angle may depend on the difference between refractive indices (n) of the LED/light guide material and the material on the other side of the interface as well as other factors (e.g., polarization).

In some examples, there may be three different types of interfaces to the tissue 335 for the wearable device in addition to the first optical paths 350 between the light sources 320 and the detectors 325 used for physiological measurements. For example, light source 320-b and light source 320-c may be embedded in the inner housing 305 (e.g., in an optically clear epoxy material such as the layer of material 345) and may emit light, such as light 330-b, coupled out of the light source optics through the layer of material 345 to the interface of the tissue 335. Similarly, the light source 320-a may be under a dome (e.g., made of epoxy) and may emit light 330-a coupled out of light source optics through the layer of material 345 to the tissue 335 interface. The light source 320-a may be flush with the layer of material 345, such that the tissue 335 may make direct contact with the light source 320-a. Further, light propagating inside the tissue 335 (e.g., finger tissue) may be coupled to the detector optics through the tissue 335 to the layer of material 345 interface.

In some examples, the wearable device may be subjected to a force or an acceleration, causing an air gap between the surface of the tissue 335 and one or more sensors at the wearable device. The air gap (e.g., with refractive index of ˜1.00) between the tissue 335 and the layer of material 345 may disturb the first optical paths 350 and the second optical paths 355, as stray light (e.g., light 340) may be coupled to the tissue 335 through two interfaces (e.g., the interface between the layer of material 345 and the air and the interface between the air and the tissue 335). Additionally or alternatively, liquid or other contaminants may be trapped between the tissue 335 and the layer of material 345. The contaminants may dampen or absorb the optical signals. Further, the difference between refractive indexes and contaminant layer absorption spectra may determine how different signal paths/channels may be affected (e.g., causing increased variability in signal strength).

Domes on top of the light sources 320, such as on top of light source 320-a, may create steeper light incidence angles in the layer of material 345 and the tissue 335 or gap interface. The domes also protrude inside relatively elastic tissue 335, improving contact. However, they may not be sufficiently large to solve disturbance to the in-coupled signal. Thus, any changes in the amount of light coupled into the tissue 335 may be compensated by controllers or drive electronics of the wearable device, causing additional losses in battery life as well as interruptions and inaccuracy to physiological measurements (e.g., PPG and SpO2 measurements). In some cases, SpO2 measurements may be affected by losses of in-coupled light and disturbance due to the oxygen saturation levels of blood being calculated as a ratio of two signals measured with two different wavelengths from light sources 320. As the two light propagation paths are both spatially and angularly different from each other, changes to either of the signal paths may cause measurement inaccuracy.

In some cases, the motion artifacts, such as force and acceleration, may result in indirect changes to the stray light that reaches the detectors 325, impacting the DC component and the AC component of the signal detected by the detectors 325 by causing dynamic noise respective to the motion artifacts. For instance, the amount of the light 340-a that propagates along the second optical path 355-a from the light source 320-a to the detector 325-a and the amount of the light 340-b that propagates along the second optical path 355-b from the light source 320-b to the detector 325-b may depend on the amount of light that is coupled to the tissue 335. When the light sources 320 lose contact with the tissue 335 (e.g., due to gaps that may be caused by motion artifacts), more stray light may be produced. For example, the light sources 320-b and 320-c may produce more stray light than the light source 320-a because the light sources 320-b and 320-c are closer to the detectors 325 than the light source 320-a. Further, because the light sources 320-b and 320-c are closer to the edges of the domes covering the detectors 325, the light sources 320-b and 320-c may lose contact with the tissue 335 more dynamically than light source 320-a since the domes may stretch the tissue 335. For instance, finger movement (e.g., during exercise) may cause a change in the perfusion index due to the changes to the stray light resulting from the changing optical interface of the layer of material 345 with the tissue 335. Additionally, the second optical paths directly from the light sources 320-b and 320-c to the detectors 325 (e.g., the second optical path 355-b) may be more direct and shorter distances than the second optical paths directly from the light source 320-a to the detectors 325 (e.g., the second optical path 355-a). Due to the proximity of the light sources 320-c and 320-b to the detectors 325 and the directness of the respective optical paths, the light sources 320-c and 320-b may produce more stray light, resulting in a greater DC background detected by the detectors 325.

Additionally or alternatively, the light sources 320-b and 320-c may produce more stray light than the light source 320-a because neither the light source 320-b nor the light source 320-c are covered by a dome. Because the light source 320-a is covered by a dome, the light source 320-a may emit light with better light incident angles for coupling of the emitted light into the tissue 335 (e.g., in-coupling) because the light incident angles are less steep. However, because the light sources 320-b and 320-c are uncovered by domes, the light sources 320-b and 320-c may emit light with steeper light incident angles, resulting in more coupling of the emitted light to the epoxy of the layer of material 345 and less in-coupling. In such cases, the lack of domes over the light sources 320-b and 320-c may also contribute to the emission of a greater amount of stray light from the light sources 320-b and 320-c than from the light source 320-a.

As described in more detail with reference to the subsequent figures, one or more physical features (e.g., light blocking features or the like) may be arranged along the inner housing 305 to block light from traveling along the one or more second optical path 355 while allowing light to travel along the one or more first optical paths 350. As such, the light blocking features may reduce the amount of stray light and/or noise entering the one or more detectors 325, thereby increasing measurement accuracy and reducing battery consumption associated with signal processing.

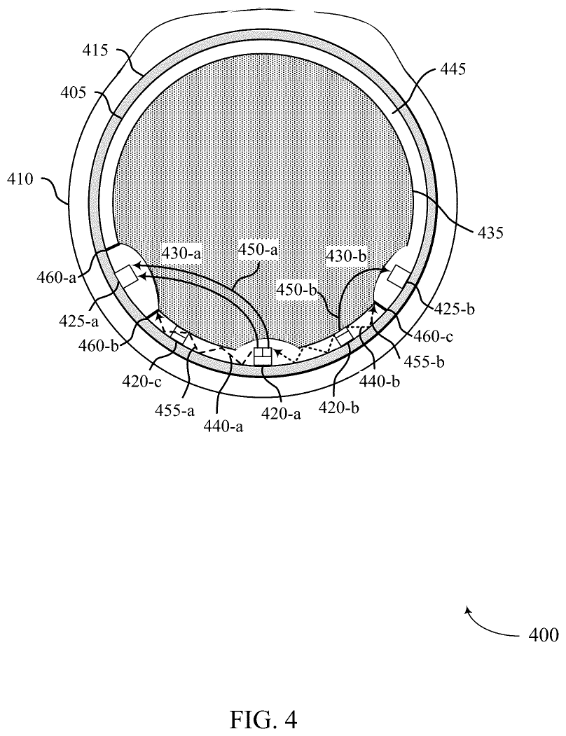

FIG. 4 illustrates an example of a wearable device diagram 400 that supports optimized structures for optical measurement in accordance with aspects of the present disclosure. The wearable device diagram 400 may implement, or be implemented by, aspects of the system 100, system 200, or both. For example, the wearable device diagram 400 may illustrate an example of a wearable device 104 as described with reference to FIG. 1. Although the wearable device is illustrated as a ring in FIG. 4, the wearable device may be any example of a wearable device (e.g., a watch, a necklace, and the like).

The wearable device in wearable device diagram 400 may include an inner housing 405, an outer housing 410, and an electronic substrate 415, which may be examples of an inner housing 305, an outer housing 310, and an electronic substrate 315 as described with reference to FIG. 3. The wearable device may include one or more light sources 420 and detectors 425, which may be examples of light sources 320 and detectors 325 as described with reference to FIG. 3. For example, light sources 420 may emit light 430 that is received by detectors 425. In this regard, the light sources 420 may support one or more first optical paths 450 through the tissue 435 for physiological data measurements. The light sources 420 may emit light 440 that is directly received by detectors 425 along one or more second optical paths 455 within the layer of material 445. In such cases, the light 440 may be an example of stray light that interferes with the efficiency and accuracy of the physiological data measurements.

In some cases, the housing (e.g., including the inner housing 405 and the outer housing 410) of the wearable device may include a ring-shaped housing having an inner circumference. For example, the wearable device may be an example of a ring. In such cases, the light sources 420 may be positioned at one or more radial positions along the inner circumference, and the one or more detectors 425 may be positioned at one or more second radial positions along the inner circumference. The second optical paths 455 may be along the inner circumference from one or more of the one or more light sources 420 to one or more of the one or more detectors 425.

In other implementations, the housing (e.g., including the inner housing 405 and the outer housing 410) of the wearing device may include a planar surface. For example, the wearable device may be an example of watch, a necklace, and the like. In such cases, the light sources 420 may be positioned at one or more first planar positions along the wearable device, and the one or more detectors 425 may be positioned at one or more second planar positions along the wearable device. The second optical paths 455 may be along the wearable device from one or more of the one or more light sources 420 to one or more of the one or more detectors 425.

The wearable device may include one or more light blocking components 460 that may be configured to block light 440 from the one or more light sources 420 along the second optical paths 455 from entering the detectors 425. The light blocking components 460 may allow light 430 from the light sources 420 along the first optical paths 450 to enter the detectors 425. In such cases, the light blocking components 460 may optically isolate detectors 425 from the light 440 and/or the rest of the inner housing 405 (e.g., other electrical circuitry of the wearable device). For instance, the light blocking component 460-a and light blocking component 460-b may be positioned on a first side of the detector 425-a and on an opposite side of the detector 425-a from the first side, respectively, such that the light 430-a enters the detector 425-a along the first optical path 450-a and light 440-a is blocked along the second optical path 455-a. Additionally, the light blocking component 460-c may be positioned on a first side of the detector 425-b such that the light 430-b enters the detector 425-b along the first optical path 450-b and light 440-b is blocked along the second optical path 455-b. In such cases, the light blocking components 460 may be disposed on the surface of the housing 405 and/or adjacent to the detectors 425.

By optically isolating the detectors 425 from the light 440, the light blocking components 460 may block optical paths 455 containing stray light (e.g., light 440). Accordingly, the light blocking components 460 may prevent stray light from reaching the detectors 425 where the stray light may reduce the quality of the physiological data measurements. For example, the light 440-a may propagate from the light source 420-a to the detector 425-a along the second optical path 455-a, and upon reaching the light blocking component 460-b, the light 440-a may dissipate, rebound, scatter, weaken, and/or be blocked due to the positioning of the light blocking component 460-b. In such cases, the light blocking component 460-b may reduce and/or eliminate the amount of the light 440-a entering the detector 425-a. Similarly, the light 440-b may propagate from the light source 420-b to the detector 425-b along the second optical path 455-b, and upon reaching the light blocking component 460-c, the light 440-b may also dissipate, rebound, scatter, weaken, and/or be blocked due to the positioning of the light blocking component 460-c. In such cases, the light blocking component 460-c may reduce and/or eliminate the amount of the light 440-b entering the detector 425-b.

In other examples, the light blocking components 460 may be disposed adjacent to the light sources 420. For instance, a light blocking component 460 may be positioned on a first side of one or more light source 420 and on an opposite side of the one or more light sources 420 from the first side such that the light 430 exits the one or more light sources 420 along the first optical path 450 to enter the detectors 425 and the light 440 is blocked along the second optical path 455. By optically isolating the light sources 420, the light blocking components 460 may block optical paths 455 containing stray light (e.g., light 440) from reaching the detectors 425. For example, the light 440 may propagate from the light source 420 along the second optical path 455 and upon reaching the light blocking component 460, the light 440 may dissipate, rebound, scatter, weaken, and/or be blocked due to the positioning of the light blocking component 460 adjacent to the light source 420. In such cases, the light blocking component 460 may reduce and/or eliminate the amount of the light 440 reaching and/or entering the detector 425.

In some cases, the light blocking components 460 may block the light 440 along the second optical paths 455 that aggregates towards a top surface of the layer of material 445. In such cases, the light blocking components 460 may block the light 440 that aggregates towards an inner surface of the layer of material 445 that is positioned adjacent to the tissue 435. For example, the light blocking components 460 may protrude through a portion of the layer of material 445 such that the light blocking components 460 block light 440 at the top of the layer of material 445. The light blocking components 460 may extend most of the way through the layer of material 445. In some cases, the light blocking components 460 may protrude through an entire portion of the layer of material 445 such that the light blocking components 460 extends from a first surface of the layer of material 445 adjacent to the inner housing 405 and to a second surface of the layer of material 445 opposite the first surface and adjacent to the tissue surface. In some cases, the layer of material 445 may include a metallic inlet shield that reduces an amount of internal stray light within the layer of material 445. For example, the layer of material 445 may be replaced by the metallic inlet shield or include, in addition to the epoxy material of the layer of material 445, the metallic inlet shield such that the metallic inlet shield reduces an amount of light 440 along the second optical path 455.

The light blocking components 460 may limit the field of view of the detectors 425, the light sources 420, or both to be towards the tissue 435 and along the first optical paths 450. For example, the light blocking components 460 may limit the field of view of the detectors 425 to be away from the light sources 420 and the second optical paths 455. In some cases, the detectors 425 may include a material disposed on a surface of the detectors 425. For example, the material may include optical properties that allows light 430 that enter the material at a first range of angles to pass through the material and blocks light 440 that enters the material at a second range of angles from passing through the material. For example, the first range of angles includes at least angles of light 430 from the first optical path 450, and the second range of angles includes at least angles of light 440 from the second optical path 455.

The light blocking components 460 may be formed from a material that may not propagate light. For example, the material may include, but is not limited to, a metallic material, an opaque material, a plastic material, a non-transparent glue or tape, an adhesive material, or a combination thereof. In some examples, the light blocking components 460 may be an example of one or more circular components at least partially surrounding the detectors 425, one or more curved walls that curve towards one or more of the detectors 425, one or more straight walls adjacent to the detectors 425, or a combination thereof. In some cases, the light blocking components 460 may be an example of one or more protrusions extending from the surface of the inner housing 405, one or more grooves in the layer of material 445 extending inward toward the surface of the inner housing 405, or both. In other examples, the light blocking components 460 may be an example of one or more circular components at least partially surrounding the light sources 420.

In some examples, a top surface of the detectors 425 may be more sensitive to light than the other surfaces of the detectors 425. In some aspects, stray light emitted directly from the light sources 420 along the second optical paths 455 may be blocked from entering the detectors 425 by raising the surface of the detectors 425 such that the stray light propagating along the second optical path 455 may not reach the light sensitive area on the top of the detectors 425. In such cases, the height of the detectors 425 may be increased during the manufacturing process. For example, the detectors 425 of the wearable devices may be manufactured to have a 0.5 mm increase in height such that the detectors 425 have a height of 1.20 mm. In some cases, the detectors 425 of the wearable device may be manufactured to include a spacer positioned between each of the detectors 425 and the PWB of the wearable device such that the top surface of the detectors extends 1.20 mm from an exposed surface of the layer of material 445. In such cases, the spacer may include a height of 0.5 mm. Although the wearable device may be manufactured to include a spacer at the interface between the bottom of the detectors 425 and the top of the PWB, changing the components for manufacturing of the wearable device may introduce difficulties in the manufacturing process.

In some examples, increasing the height of the detectors 425 may reduce an amount of stray light received by the detectors 425 to be 99% less stray light (e.g., along the second optical path 455) compared to a wearable device that fails increase the height of the detectors 425. In such cases, increasing the height of the detectors 425 by 0.5 mm may allow 1% of stray light to enter the detectors 425 as compared to a wearable that does not increase the height of the detectors 425. In some cases, the DC power of the PPG signal may decrease, thereby increasing a power consumption associated with the light sources 420 or other components of the wearable device and decreasing a battery life of the wearable device. For example, the DC power consumption may include 90% of power for blood oxygen saturation and heart rate measurements using IR light (e.g., via light source 420-a) compared to a wearable device that does not increase the height of the detectors 425. In other examples, the DC power consumption may include 45% of power for heart rate measurements using green light (e.g., via light source 420-b and light source 420-c) compared to a wearable device that does not increase the height of the detectors 425.

In some cases, increasing the height of the detectors 425 by 0.5 mm may decrease an amount of stray light from entering the detectors 425 along the second optical path without significantly decreasing the DC signal component. Accordingly, raising the height of the detectors 425 may increase the perfusion index, thereby increasing the quality of the PPG. In some cases, raising the height of the detectors 425 may increase the quality of the SpO2 signal. For example, the perfusion index may increase 2.3 times for blood oxygen saturation and heart rate measurements using IR light (e.g., via light source 420-a). In other examples, the perfusion index may increase 2.0 times for heart rate measurements using green light (e.g., via light source 420-b and light source 420-c). The increased height of the detectors 425 may improve the perfusion index, decrease the DC signal component, and increase the quality of the SpO2 signal.

The wearable device may obtain oxygen saturation measurements (e.g., SpO2 measurements) by using the light (e.g., red and IR light) emitted from the light source 420-a, and the wearable device may also obtain PPG measurements (e.g., heart rate measurements) by using the light (e.g., green light) emitted from the light source 420-b and the light source 420-c. The stray light may include light emitted from the light source 420-a, the light source 420-b, and the light source 420-c. Accordingly, the stray light may include aspects of red light, IR light, and green light. By blocking the stray light (e.g., light 440), the light blocking components 460 may increase the quality of the SpO2 signal, the PPG signal, or both. For instance, the DC component of the stray light may be significantly larger than the AC component of the stray light. Because PPG measurements such as heart rate measurements are based on the AC component of the signal, the PPG measurements may be more susceptible to changes in the AC component of the signal. Because the SpO2 measurements are based on the perfusion index, the SpO2 measurements may be susceptible to changes in the ratio between the AC component and the DC component. Accordingly, the SpO2 measurements may be more affected by stray light than the PPG measurements.

The light blocking components 460 may increase the quality of the signal used by the wearable device to perform PPG and SpO2 measurements by blocking the stray light, although blocking the stray light may have a more drastic effect to SpO2 measurements as compared to the heart rate measurements. During periods of time in which the wearable device is in motion (e.g., during an activity period), the stray light may include increased amounts of green light. Because the PPG measurements may be obtained using green light emitted from the light source 420-b and the light source 420-c, the light blocking components 460 may increase the quality of PPG measurements during periods of movement.

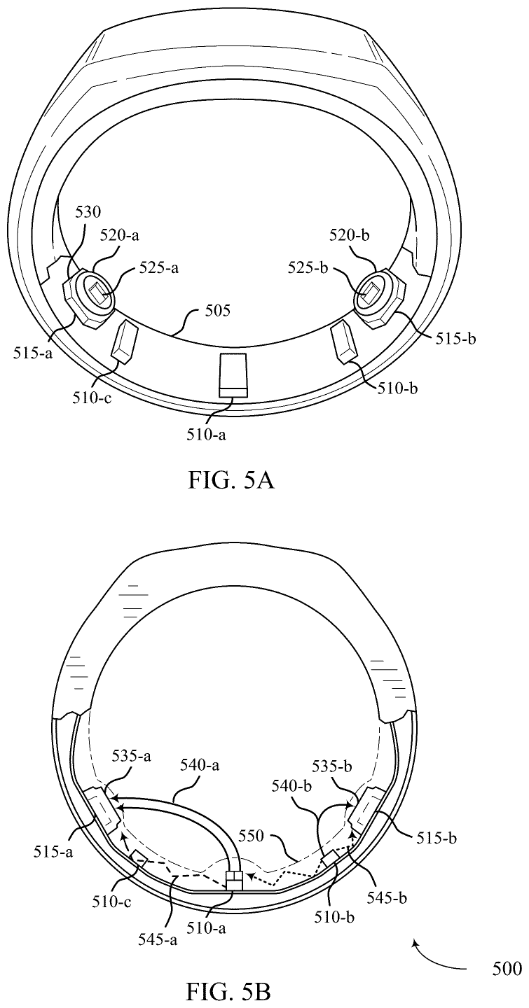

FIG. 5A illustrates an example of a perspective view of a wearable device 500 that supports optimized structures for optical measurement in accordance with aspects of the present disclosure. The wearable device 500 may implement, or be implemented by, aspects of the system 100, system 200, wearable device diagram 300, wearable device diagram 400, or a combination thereof. For example, wearable device 500 may illustrate examples of wearable devices 104 as described with reference to FIGS. 1-4. Although the wearable device 500 is illustrated as a ring in FIG. 5, aspects and components of the wearable device 500 illustrated in FIG. 5 may be implemented in any type of wearable device (e.g., a watch, a bracelet, a necklace, and the like).

In some examples, the wearable device 500 may contain housing 505 which may be an example of the housing as described with reference to FIGS. 2-4. One or more sensors may be embedded in the housing, such as one or more light sources (e.g., LEDs) for collecting physiological measurements (e.g. physiological data) from the user. As described with reference to FIGS. 3 and 4, light source 510-b and light source 510-c, which may be green LEDs, may emit light, such that the light may be guided along to detector 515-a and detector 515-b along the first optical path 540 and the second optical path 545. In some cases, detector 515-a, detector 515-b, or both may detect light emitted from one or more light sources 510, such as light source 510-c and light source 510-b, respectively, and one or more additional light sources 510 (e.g., light source 510-a) used for physiological measurements. For example, light source 510-a, which may be a red/IR LED, may emit light, such that the light may be guided along to detector 515-a and detector 515-b along the first optical path 540 and the second optical path 545.

The wearable device 500 may include the light blocking components 520. The light blocking components 520 may be an example of a light blocking component as described with reference to FIGS. 1-4. In accordance with various examples, light blocking components 520 may be used to block stray light along the second optical path 545 and allow light along the first optical path 540 to enter the detectors 515, as described with reference to FIG. 4.

The light blocking component 520-a may be disposed adjacent to the detector 515-a, and the light blocking component 520-b may be disposed adjacent to the detector 515-b. For example, the light blocking component 520-a may be disposed around the detector 515-a such that the light blocking component 520-a encompasses the detector 515-a. In some examples, the light blocking component 520-a and light blocking component 520-b may be examples of plastic molds at least partially surrounding the detector 515-a and detector 515-b, respectively. For example, the light blocking component 520-a and light blocking component 520-b may fully surround the detector 515-a and detector 515-b, respectively. The light blocking components 520 may include one or more plastic materials that may include, but are not limited to, polyethylene, polypropylene, polyethylene terephthalate, or a combination thereof.

In some implementations, the light blocking components 520 may be examples circular components at least partially surrounding the detectors 515. The circular components may include an opening 525 which may include an inner cavity of the circular components. The opening 525 of the light blocking components 520 may be configured to expose the one or more detectors 515 to the light traveling through the tissue surface. For example, the opening 525 of the light blocking components 520 may be configured to expose the detectors 515 to the light from the light sources 510 along the first optical path 540. In some cases, the opening 525 may be configured to expose one or more corners of the detectors 515 to the light along the first optical path 540. In such cases, the corners of the detectors 515 may receive light from the light sources 510 at least partially through the tissue surface.

The circular components may include a wall 530 that blocks light from the light sources 510 along the second optical path 545. In such cases, the wall 530 may block the light from entering the detectors 515. In some examples, the light blocking components 520 (e.g., including the wall 530) may block the one or more corners from the stray light traveling directly from the light sources 510 along the second optical path 545. The light blocking components 520 may separate (e.g., isolate) the detectors 515 from the electronic circuitry of the wearable device 500 (e.g., including the light sources 510). In such cases, the light blocking components 520 may limit the field of view of the detectors 515 to be straight forward and towards the tissue surface along the first optical path 540 rather than to the sides of the detectors 515 (e.g., towards the light sources 510 along the second optical path 545).

The circular components may be an example of an injection molded piece that is placed on top of the detectors 515 during manufacturing. For example, the light blocking components 520 may include injection molded plastic pieces that are glued over the detectors 515. The light blocking components 520 may be an example of protective pieces to block the direct from the light sources 510 from entering the detectors 515 along the second optical path 545. For example, the light blocking components 520 may be glued on top of the detectors 515 under the domes. The manufacturing of the light blocking components 520 may include a lower cost than manufacturing of other light blocking components, but includes added complexity during assembly.

The light blocking components 520 may include a wall thickness that affects the material light transmittance. For example, the length of the light blocking component 520 may be 4.22 mm, the width may be 3.00 mm, and the height may be 1.39 mm. In such cases, the height of the wall 530 may be 1.39 mm. The inner diameter of the opening 525 may be 2.33 mm. The diameter of the outside rim of the opening 525 may be 2.90 mm. The diameter of the opening 525 may enable the light blocking component 520 to expose a larger surface area of the detectors 515 to the light along the first optical path 540. In such cases, the opening 525 may include a wider diameter that exposes the light sensitive surface corners of the detectors 515, thereby enabling a stronger signal of the PPG DC signal component and mitigating the PPG DC signal blocking feature.

FIG. 5B illustrates a cross-sectional view of the wearable device 500 in accordance with aspects of the present disclosure. As previously described with respect to FIGS. 3 and 4, the detectors 515 may receive light emitted from the one or more light sources 510 along a first optical path 540 (e.g., through the tissue surface) and a second optical path 545 (directly from the one or more light sources 510). In such cases, the wearable device 500 may include light blocking components 520.

The light blocking components 520 may include one or more protrusions 535. The one or more protrusions 535 may be an example of the wall 530 of the circular component. The protrusions 535 may extend from the surface of the housing 505. For example, the protrusions 535 may extend 1.39 mm from the surface of the housing 505. The protrusions 535 may be configured to optically isolate the detectors 515 from the material 550 disposed on the surface of the housing 505, the light sources 510, other electrical circuitry of the wearable device 500, or a combination thereof. In some examples, the protrusions 535 may be configured to optically isolate the detectors 515 from the light from the light sources 510 along the second optical path 545 within the layer of material 550. The layer of material 550 may be an example of a layer of material as described with reference to FIGS. 3 and 4. In some cases, the protrusions 535 of the light blocking components 520 may separate (e.g., isolate) the detectors 515 from the electronic circuitry of the wearable device 500. In such cases, the protrusions 535 may limit the field of view of the detectors 515 to be straight forward and towards the tissue surface (e.g., along the first optical path 540) rather than to the sides of the detectors 515 (e.g., along the second optical path 545).

In some cases, the light emitted from light source 510-a may propagate near a surface of the layer of material 550 (e.g., close to the epoxy surface). For example, the light emitted from light source 510-a may propagate near the outer edges of the material 550 rather than propagating through the center of the material 550. In such cases, the protrusions 535 of the light blocking components 520 may block the stray light emitted from the light source 510-a (as well as light source 510-b and light source 510-c) more efficiently by the protrusions 535 extending from the surface of the housing 505 and through the material 550 such that the detectors 515 are optically isolated from the light sources 510. For example, the protrusions 535 may protrude at least partially (e.g., most of the way) through the material 550 (e.g., epoxy layer) such that the protrusions 535 of the light blocking components 520 block the light traveling at the top of the material 550 along the second optical path 545. In some cases, the light blocking components 520 may limit the risk of error in blood oxygen saturation measurements and optical interface changes (e.g., caused by movement of the user, etc.).

The light blocking components 520 may reduce an amount of stray light received by the detectors 515 to be 99% less stray light (e.g., along the second optical path 545) compared to a wearable device that fails to include one or more light blocking components 520. In such cases, the light blocking components 520 may allow 1% of stray light to enter the detector 515 as compared to a wearable device that does not include a light blocking component 520. In some cases, the DC power of the PPG signal may decrease, thereby increasing a power consumption associated with the light sources 510 or other components of the wearable device 500 and decreasing a battery life of the wearable device 500. For example, the DC power consumption may include 46-50% of power for blood oxygen saturation and heart rate measurements using IR light (e.g., via light source 510-a) compared to a wearable device that does not include a light blocking component 520. In other examples, the DC power consumption may include 13-15% of power for heart rate measurements using green light (e.g., via light source 510-b and light source 510-c) compared to a wearable device that does not include a light blocking component 520.

In some cases, the use of the light blocking components 520 may increase the perfusion index, thereby increasing the quality of the PPG signal. For example, the perfusion index may increase 2.1-2.3 times for blood oxygen saturation and heart rate measurements using IR light (e.g., via light source 510-a). In other examples, the perfusion index may increase 3 times for heart rate measurements using green light (e.g., via light source 510-b and light source 510-c). The light blocking components 520 may improve the perfusion index, decrease the DC signal component, and increase the quality of the overall signal. In such cases, the light blocking components 520 may increase stray light suppression by decreasing a level of epoxy stray light below 1% in both optical channels (e.g., the first optical path 540 and the second optical path 545).

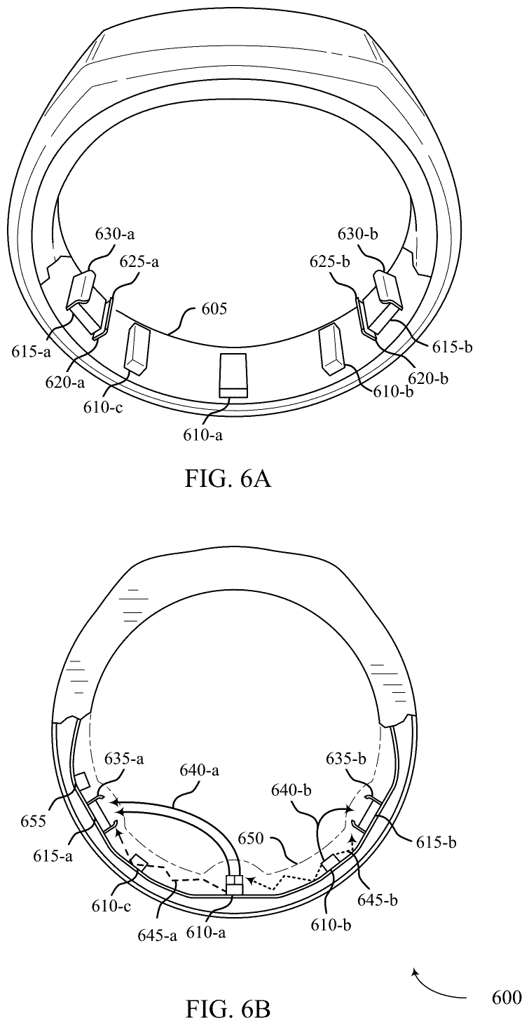

FIG. 6A illustrates an example of a perspective view of a wearable device 600 that supports optimized structures for optical measurement in accordance with aspects of the present disclosure. The wearable device 600 may implement, or be implemented by, aspects of the system 100, system 200, wearable device diagram 300, wearable device diagram 400, wearable device 500, or a combination thereof. For example, wearable device 600 may illustrate examples of wearable devices 104 as described with reference to FIGS. 1-4. Although the wearable device 600 is illustrated as a ring in FIG. 6, aspects and components of the wearable device 600 illustrated in FIG. 6 may be implemented in any type of wearable device (e.g., a watch, a bracelet, a necklace, and the like).

The wearable device 600 may contain housing 605 which may be an example of the housing as described with reference to FIGS. 2-5. The housing 605 may contain one or more embedded sensors, such as one or more light sources 610 (e.g., LEDs) for collecting physiological measurements (e.g., physiological data) from the user. As described with reference to FIGS. 3 and 4, the light sources 610 may emit light, such that the light may be guided along to detectors 615 along the first optical path 640 and the second optical path 645.

The wearable device 600 may include the light blocking components 620. The light blocking components 620 may be an example of a light blocking component as described with reference to FIGS. 1-5. In accordance with various examples, the light blocking components 620 may be used to block stray light along the second optical path 645 from entering the detectors 615 and allow light along the first optical path 640 to enter the detectors 615, as described with reference to FIG. 4.

The light blocking components 620 may include a first wall 625 and a second wall 630 opposite of the first wall 625. For example, the light blocking component 620-a may include a first wall 625-a and a second wall 630-a, and the light blocking component 620-b may include a first wall 625-b and a second wall 630-b. The first walls 625 and the second walls 630 may extend from the surface of the housing 605 and curve towards the detectors 615. For example, the first walls 625 and the second walls 630 may include curved portions that curve towards the detectors 615. In such cases, the first wall 625 and the second wall 630 may form a partial dome over the detectors 615.

The light blocking components 620 may be disposed adjacent to the detectors 615. For example, the first wall 625-a of light blocking component 620-a may be disposed next to the detector 615-a such that the first wall 625-a of light blocking component 620-a is positioned between the detector 615-a and the light sources 610. The first wall 625-b of light blocking component 620-b may be disposed next to the detector 615-b such that the first wall 625-b of the light blocking component 620-b is positioned between the detector 615-b and the light sources 610. For example, the light blocking component 620-a may form a barrier between the detector 615-a and the light sources 610, and the light blocking component 620-b may form a barrier between the detector 615-b and the light sources 610. The second walls 630 of light blocking components 620 may be disposed next to the detectors 615 such that the second walls 630 of the light blocking components 620 are positioned between the detectors 615 and other circuitry 655 of the wearable device. In some examples, the light blocking components 620 may separate (e.g., optically isolate) the detectors 615 from circuitry 655 of the wearable device, the light sources 610, or both.

The light blocking components 620 (including at least the first wall 625 and the second wall 630) may block light from entering the detectors 615 along the second optical path 645 and allow light to enter the detectors 615 along the first optical path 640. In such cases, the light blocking components 620 may be configured to expose the one or more detectors 615 to the light traveling through the tissue surface from the light sources 610 along the first optical path 640. In such cases, the light blocking components 620 may limit the field of view of the detectors 615 to be towards the tissue surface along the optical path 640 rather than to one or more sides of the detectors 615 towards the light sources 610 along the second optical path 645. In some examples, the light blocking component 620-a and light blocking component 620-b may be examples of molded pieces at least partially obstructing a field of view of the detector 615-a and detector 615-b, respectively.

The first wall 625 and the second wall 630 of the light blocking components 620 may be connected (e.g., bonded) to the PWB around the detectors 615 during manufacturing. The light blocking components 620 may include one or more metal materials that may include, but are not limited to, aluminum, aluminized steel, copper, carbon steel, or a combination thereof. For example, the light blocking components 620 may include sheet metal pieces that are soldered or glued onto the PWB adjacent to the detectors 615. The light blocking components 620 may be an example of protective pieces to block the direct light from the light sources 610 from entering the detectors 615 along the second optical path 645. For example, the light blocking components 620 may be glued onto the PWB between the light sources 610 and detectors 615 under the domes. The manufacturing of the light blocking components 620 may include a lower cost of manufacturing than other light blocking components, but includes added complexity in assembly of the PWB.

The light blocking components 620 may include a wall thickness that affects the material light transmittance. In some examples, the electronic components on either side of the detectors 615 on the PWB, such as the light sources 610 and the circuitry 655, may have different heights, lengths, or both. The light blocking components 620 may include a length and a height that affects the amount of light that is blocked from the detectors 615 along the second optical path 645. For example, the first wall 625 and the second wall 630 of the light blocking component 620 may each have a length of 3.33 mm and a height of 1.49 mm. In some examples, the height of the second wall 630 may be different from the height of the first wall 625. The thickness of the first wall 625 and the second wall 630 may be 0.20 mm. In some implementations, the distance between the first wall 625 and the second wall 630 may be 2.90 mm. In some examples, the distance between the first wall 625 and the second wall 630 may enable the light blocking component 620 to expose a larger surface area of the detectors 615 to the light along the first optical path 640.

FIG. 6B illustrates a cross-sectional view of the wearable device 600 in accordance with aspects of the present disclosure. As previously described with respect to FIGS. 3 and 4, detector 615-a and detector 615-b may receive light emitted from the one or more light sources 610 along a first optical path 640 (e.g., through the tissue surface) and a second optical path 645 (directly from the one or more light sources 610). In such cases, the wearable device 600 may include a light blocking components 620.

The light blocking components 620 may include one or more protrusions 635. The one or more protrusions 635 may be an example of the first wall 625, the second wall 630, or both. The protrusions 635 may extend from the surface of the housing 605. For example, the protrusions 635 may extend 1.49 mm from the surface of the housing 605. The protrusions 635 may be configured to optically isolate the detectors 615 from the material 650 disposed on the surface of the housing 605, the light sources 610, other electrical circuitry 655 of the wearable device 600, or a combination thereof. In some examples, the protrusions 635 may be configured to optically isolate the detectors 515 from the light emitted from the light sources 610 along the second optical path 645 within the layer of material 650. The layer of material 650 may be an example of a layer of material as described with reference to FIGS. 3 and 4.

In some cases, the light emitted from light source 610-a may propagate near a surface of the layer of material 650 (e.g., close to the epoxy surface). For example, the light emitted from light source 610-a may propagate near the outer edges of the material 650 rather than propagating through the center of the material 650. In such cases, the protrusions 635 of the light blocking components 620 may block the stray light emitted from the light source 610-a (as well as light source 610-b and light source 610-c) more efficiently by the protrusions extending from the surface of the housing 505 and through the material 650 (e.g., epoxy layer) such that the protrusions 635 of the light blocking components 620 block the light traveling at the top of the material 650 along the second optical path 645-c.). Accordingly, the light blocking components 620 may reduce the DC component of the signal detected by the detectors 615 less than the light blocking components 520, contributing to less power consumption by the wearable device 600 compared to the wearable device 500.

The light blocking components 620 may reduce an amount of stray light received by the detectors 615 to be 85% less stray light (e.g., along the second optical path 645) compared to a wearable device that fails to include one or more light blocking components 620. In such cases, the light blocking components 620 may allow 15% of stray light to enter the detectors 615 as compared to a wearable device that does not include a light blocking component 620. In some cases, the DC power of the PPG signal may decrease, thereby increasing a power consumption associated with the light sources 610 or other components of the wearable device 600 and decreasing a battery life of the wearable device 600. For example, the DC power consumption may include 61% of power for blood oxygen saturation and heart rate measurements using IR light (e.g., via light source 610-a) compared to a wearable device that does not include a light blocking component 620. In other examples, the DC power consumption may include 15% of power for heart rate measurements using green light (e.g., via light source 610-b and light source 610-c) compared to a wearable device that does not include a light blocking component 620.

In some cases, the use of the light blocking components 620 may increase the perfusion index, thereby increasing the quality of the PPG signal. For example, the perfusion index may increase 2.1 times for blood oxygen saturation and heart rate measurements using IR light (e.g., via light source 610-a). In other examples, the perfusion index may increase 2.7 times for heart rate measurements using green light (e.g., via light source 610-b and light source 610-c). The light blocking components 620 may improve the perfusion index, decrease the DC signal component, and increase the quality of the overall signal.

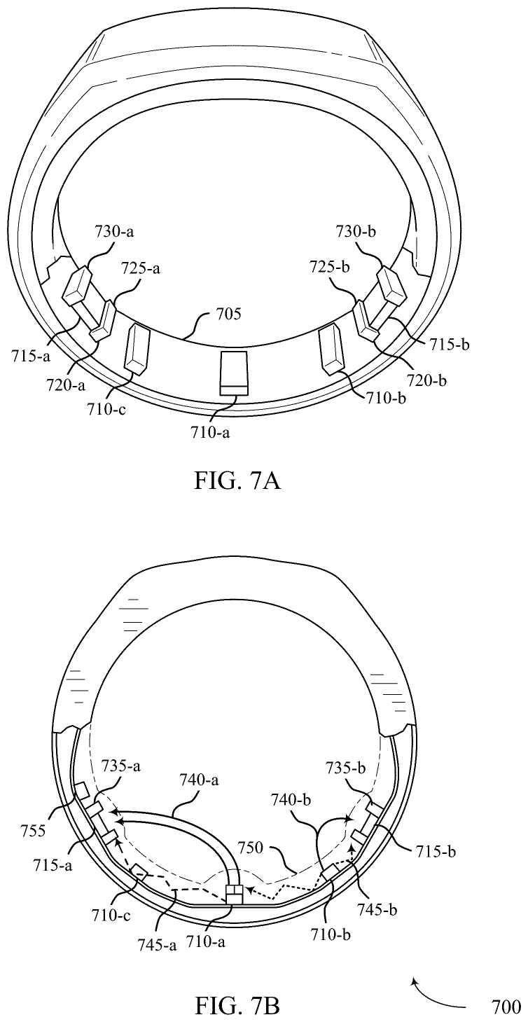

FIG. 7A illustrates an example of a perspective view of a wearable device 700 that supports optimized structures for optical measurement in accordance with aspects of the present disclosure. The wearable device 700 may implement, or be implemented by, aspects of the system 100, system 200, wearable device diagram 300, wearable device diagram 400, wearable device 500, wearable device 600, or a combination thereof. For example, wearable device 700 may illustrate examples of wearable devices 104 as described with reference to FIGS. 1-4. Although the wearable device 700 is illustrated as a ring in FIG. 7, aspects and components of the wearable device 700 illustrated in FIG. 7 may be implemented in any type of wearable device (e.g., a watch, a bracelet, a necklace, and the like).

The wearable device 700 in wearable may contain housing 705 which may be an examples of the housing as described with reference to FIGS. 2-6. The housing 705 may contain one or more embedded sensors, such as one or more light sources 710 (e.g., LEDs) for collecting physiological measurements (e.g., physiological data) from the user. As described with reference to FIGS. 3 and 4, the light sources 710 may emit light, such that the light may be guided along to detectors 715 along the first optical path 740 and the second optical path 745.

The wearable device 700 may include the light blocking components 720. The light blocking components 720 may be an example of a light blocking component as described with reference to FIGS. 1-4. In accordance with various examples, the light blocking components 720 may be used to block stray light along the second optical path 745 from entering the detectors 715 and allow light along the first optical path 740 to enter the detectors 715, as described with reference to FIG. 4.

The light blocking components 720 may include a first wall 725 and a second wall 730 opposite of the first wall 725. For example, the light blocking component 720-a may include a first wall 725-a and a second wall 730-a, and the light blocking component 720-b may include a first wall 725-b and a second wall 730-b. The first walls 725 and the second walls 730 may extend from the surface of the housing 705 in a linear (e.g., straight) fashion without curving towards or away from the detectors 715. For example, the first walls 725 and the second walls 730 may include unbending portions that extend straight from the surface of the housing 705. In such cases, the first wall 725 and the second wall 730 may form straight barriers (e.g., partitions) next to the detectors 715.

As described with reference to FIG. 6, the first walls 725 and the second walls 730 may be disposed adjacent to the detectors 715. The first walls 725 of the light blocking components 720 may be positioned between the detectors 715 and the light sources 710, and the second walls 730 of the light blocking components 720 may be positioned between the detectors 715 and other circuitry 755 of the wearable device 700. In some examples, the light blocking components 720 may separate (e.g., optically isolate) the detectors 715 from circuitry 755 of the wearable device, the light sources 710, or both.

As further described with reference to FIG. 6, the light blocking components 720 may block light from entering the detectors 715 along the second optical path 745 and allow light to enter the detectors 715 along the first optical path 740 through the tissue surface. In such cases, the light blocking components 720 may limit the field of view of the detectors 715 to be towards the tissue surface along the optical path 740 rather than to one or more sides of the detectors 715 (e.g., 615 towards the light sources 710 along the second optical path 745). In some aspects, the light blocking component 720-a and the light blocking component 720-b may include a material that at least partially obstructs the field of view of the detector 715-a and the detector 715-a, respectively.

The first walls 725 and the second walls 730 of the light blocking components 720 may be connected (e.g., bonded) to the PWB adjacent to the detectors 715 during manufacturing. For example, the first wall 725 and the second wall 730 of the light blocking components 720 may be disposed on top of the PWB and one or more electrical components of the wearable device 700 during manufacturing. The light blocking components 720 may include materials that may include, but are not limited to, non-transparent materials, ultraviolet (UV) curable glue (e.g., UV cure acrylates), an adhesive material (e.g., tape), or a combination thereof. For example, the first walls 725 and the second walls 730 of the light blocking components 720 may be molded from non-transparent glue. In such cases, the light blocking components 720 may be glued onto the PWB adjacent to the detectors 715. In some examples, the light blocking components 720 may be soldered or bonded to the PWB by applying UV light to the light blocking components 720 as the light blocking components 720 are positioned adjacent to the detectors 715. In some cases, the light blocking components 720 may be an example of adhesive structures or materials that are taped onto the PWB adjacent to the detectors 715. Manufacturing the light blocking components 720 with an adhesive, non-transparent material may include a lower cost of manufacturing than other light blocking components, as well as reduced complexity in the manufacturing process.

As described with reference to FIG. 6, the light blocking components 720 may include a wall thickness that affects the material light transmittance properties. In some examples, the first walls 725 and the second walls 730 of the light blocking component 720 may each have a length of 3.50 mm and a thickness of 0.70 mm. In some implementations, the distance between the first wall 725 and the second wall 730 of the light blocking component 720 may be 2.10 mm. In some examples, the distance between the first wall 725 and the second wall 730 may enable the light blocking component 720 to expose a larger surface area of the detectors 715 to the light along the first optical path 740.

FIG. 7B illustrates a cross-sectional view of the wearable device 700 in accordance with aspects of the present disclosure. As previously described with respect to FIGS. 3 and 4, detector 715-a and detector 715-b may receive light emitted from the one or more light sources 710 along a first optical path 740 (e.g., through the tissue surface) and a second optical path 745 (directly from the one or more light sources 710). In such cases, the wearable device 700 may include a light blocking components 720.

As discussed with reference to FIG. 6, the light blocking components 720 may include one or more protrusions 735 which may extend from the surface of the housing 705. The one or more protrusions 735 may be an example of the first wall 725, the second wall 730, or both. The protrusions 735 may be configured to optically isolate the detectors 715 from the material 750 disposed on the surface of the housing 705, the light sources 710, other electrical circuitry 755 of the wearable device 700, or a combination thereof. The layer of material 750 may be an example of a layer of material as described with reference to FIGS. 3-6. For example, the protrusions 735 may optically isolate the detectors 715 from the light sources 710 by interfering with and blocking the light traveling along the second optical paths 745. In such cases, the protrusions may block the stray light emitted from the light sources 710 along the second optical path 745 from entering the detectors 715.

The light blocking components 720 may reduce an amount of stray light received by the detectors 715 to be 87% less stray light (e.g., along the second optical path 745) compared to a wearable device that fails to include one or more light blocking components 720. In such cases, the light blocking components 720 may allow 13% of stray light to enter the detectors 715 as compared to a wearable device that does not include a light blocking component 720. In some cases, the DC power of the PPG signal may decrease, thereby increasing a power consumption associated with the light sources 710 or other components of the wearable device 700 and decreasing a battery life of the wearable device 700. For example, the DC power consumption may include 83% of power for blood oxygen saturation and heart rate measurements using IR light (e.g., via light source 710-a) compared to a wearable device that does not include a light blocking component 720. In other examples, the DC power consumption may include 37% of power for heart rate measurements using green light (e.g., via light source 710-b and light source 710-c) compared to a wearable device that does not include a light blocking component 720.

In some cases, the use of the light blocking components 720 may increase the perfusion index, thereby increasing the quality of the PPG signal. For example, the perfusion index may increase 1.8 times for blood oxygen saturation and heart rate measurements using IR light (e.g., via light source 710-a). In other examples, the perfusion index may increase 2.5 times for heart rate measurements using green light (e.g., via light source 710-b and light source 710-c). The light blocking components 720 may improve the perfusion index, decrease the DC signal component minimally, and increase the quality of the overall signal.

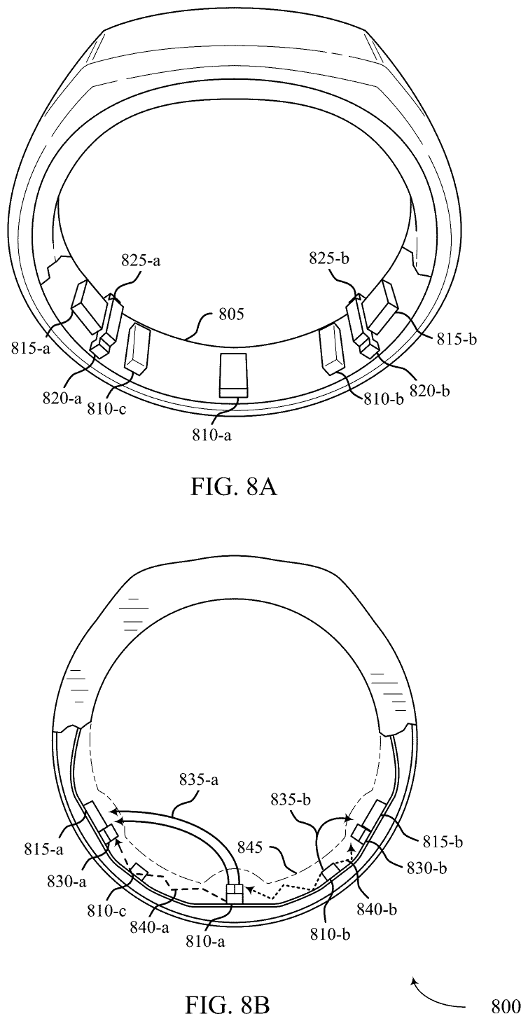

FIG. 8A illustrates an example of a perspective view of a wearable device 800 that supports optimized structures for optical measurement in accordance with aspects of the present disclosure. The wearable device 800 may implement, or be implemented by, aspects of the system 100, system 200, wearable device diagram 300, wearable device diagram 400, wearable device 500, wearable device 600, wearable device 700, or a combination thereof. For example, wearable device 800 may illustrate examples of wearable devices 104 as described with reference to FIGS. 1-4. Although the wearable device 800 is illustrated as a ring in FIG. 8, aspects and components of the wearable device 800 illustrated in FIG. 8 may be implemented in any type of wearable device (e.g., a watch, a bracelet, a necklace, and the like).

The wearable device 800 may contain housing 805 which may be an example of the housing as described with reference to FIGS. 2-7. The housing 805 may contain one or more embedded sensors, such as one or more light sources 810 (e.g., LEDs) for collecting physiological measurements (e.g., physiological data) from the user. As described with reference to FIGS. 3 and 4, the light sources 810 may emit light, such that the light may be guided along to detectors 815 along the first optical path 835 and the second optical path 840.

The wearable device 800 may include the light blocking components 820. The light blocking components 820 may be an example of a light blocking component as described with reference to FIGS. 1-4. In accordance with various examples, the light blocking components 820 may be used to block stray light along the second optical path 840 from entering the detectors 815 and allow light along the first optical path 835 to enter the detectors 815, as described with reference to FIG. 4. The light blocking components 820 may include a wall 825. For example, the light blocking component 820-a may include a wall 825-a, and the light blocking component 820-b may include a wall 825-b. The walls 825 may extend from the surface of the housing 805. For example, the walls 825 may extend from the surface of the housing 805 in a linear (e.g., straight) fashion without curving towards or away from the detectors 815. In such cases, the first walls 825 may form straight barriers (e.g., partitions) next to the detectors 815.

The walls 825 of the light blocking components 820 may include a stair-stepped configuration that extends from a first side of the housing 805 to a second side of the housing 805 opposite of the first side. For example, the walls 825 may include one or more lateral plateau regions on each side of the light blocking component 820. In some cases, the walls 825 may include a single lateral plateau region either side of the light blocking component 820. In such cases, the walls 825 may include a bridged portion that connects a first lateral plateau region on a first side of the light blocking component 820 to a second lateral plateau region on a second side of the light blocking component 820 opposite of the first side. In some examples, the light blocking components 820 may be an example of the light blocking components as described with reference to FIGS. 6 and 7. For example, the light blocking components 820 may include a curved portion that curves inwards towards the detectors 815 to form at least a partial dome over one side of the detectors 815. In other examples, the light blocking components 820 may include unbending portions that extend straight from the surface of the housing 805.

The light blocking components 820 may be disposed adjacent to the detectors 815. For examples, the walls 825 of the light blocking components 820 may be positioned between the detectors 815 and the light sources 810 of the wearable device. In some examples, the light blocking components 820 may separate (e.g., optically isolate) the detectors 815 from the light sources 810. In some examples, the light blocking components 820 may block light from entering the detectors 815 along the second optical path 840 and allow light to enter the detectors 815 along the first optical path 835 through the tissue surface. In such cases, the light blocking components 820 may limit the field of view of the detectors 815 to be towards the tissue surface along the optical path 835 rather than to the sides of the detectors 815 that are facing towards the light sources 810. In some aspects, the light blocking component 820-a and the light blocking component 820-b may include a material that at least partially obstructs a field of view of the detector 815-a and the detector 815-b, respectively.

The walls 825 of the light blocking components 820 may be connected (e.g., bonded) to the PWB adjacent to the detectors 815 during manufacturing. For example, the walls 825 of the light blocking components 820 may be disposed on top of the PWB and one or more electrical components of the wearable device 800 during manufacturing. In some cases, the walls 825 of the light blocking components 820 may be taped to the PWB between the light sources 810 and the detectors 815 during manufacturing. The light blocking components 820 may include a material that may include, but is not limited to, an adhesive material such as tape (e.g. capton tape) or other type of adhesive films. For examples, the walls 825 may be formed from strips of capton tape with adhesive surfaces. The manufacturing of the light blocking components 820 may include a lower cost of manufacturing than other light blocking components, as well as increased simplicity in the manufacturing process.

The electrical components on either side of the detectors 815 (e.g., including the light sources 810) may have different heights and lengths. Because the heights and lengths of the various electrical components of the wearable device 800 may vary, the light blocking components 820 may include a height and length that affects the amount of light that is blocked from entering the detectors 815 along the second optical path 840. For example, the light blocking components 820 may have a height of 0.97 mm and a length of 6.26 mm. In some implementations, the thickness of the wall 825 may be 1.00 mm.

FIG. 8B illustrates a cross-sectional view of the wearable device 800 in accordance with aspects of the present disclosure. As previously described with respect to FIGS. 3 and 4, detector 815-a and detector 815-b may receive light emitted from the one or more light sources 810 along a first optical path 835 (e.g., through the tissue surface) and a second optical path 840 (directly from the one or more light sources 810). In such cases, the wearable device 800 may include a light blocking components 820.

The light blocking components 820 may include a protrusion 830. For example, the light blocking component 820-a may include a protrusion 830-a, and the light blocking component 820-b may include a protrusion 830-b. The protrusion 830-a may be an example of the wall 825-a, and the protrusion 830-b may be an example of the wall 825-b. As described with reference to FIGS. 6 and 7, the protrusions 830 may extend from the surface of the housing 805. For example, the protrusions 830 may extend 0.97 mm from the surface of the housing 805. The protrusions 830 may be configured to optically isolate the detectors 815 from the material 845 disposed on the surface of the housing 805, the light sources 810, or both. For example, the protrusions 830 may be configured to optically isolate the detectors 815 from the light emitted from the light sources 810 along the second optical path 840 within the layer of material 845. The layer of material 845 may be an example of a layer of material as described with reference to FIGS. 3 and 4.

The stray light that travels along the second optical path 840 from the light sources 810 may enter the detectors 815 at a near side of the detectors 815 closest to the light sources 810. Therefore, including a one-sided taped wall, such as the wall 825 between the light sources 810 and the detectors 815, in each of the light blocking components 820 may provide adequate blocking of stray light along the second optical path 840 as well as reduced complexity in assembly as compared to the assembly of light blocking components described with reference to FIGS. 5-7.

The light blocking components 820 may reduce an amount of stray light received by the detectors 815 to be 84% less stray light (e.g., along the second optical path 840) compared to a wearable device that fails to include one or more light blocking components 820. In such cases, the light blocking components 820 may allow 14% of stray light to enter the detectors 815 as compared to a wearable device that does not include a light blocking component 820. In some cases, the DC power of the PPG signal may decrease, thereby increasing a power consumption associated with the light sources 810 or other components of the wearable device 800 and decreasing a battery life of the wearable device 800. For example, the DC power consumption may include 85% of power for blood oxygen saturation and heart rate measurements using IR light (e.g., via light source 810-a) compared to a wearable device that does not include a light blocking component 820. In other examples, the DC power consumption may include 42% of power for heart rate measurements using green light (e.g., via light source 810-b and light source 810-c) compared to a wearable device that does not include a light blocking component 820.

In some cases, the use of the light blocking components 820 may increase the perfusion index and increase the quality of the PPG signal. For example, the perfusion index may increase 1.4 times for blood oxygen saturation and heart rate measurements using IR light (e.g., via light source 810-a). In other examples, the perfusion index may increase 2.3 times for heart rate measurements using green light (e.g., via light source 810-b and light source 810-c). The light blocking components 820 may improve the perfusion index, decrease the DC signal component, and increase the quality of the overall signal.

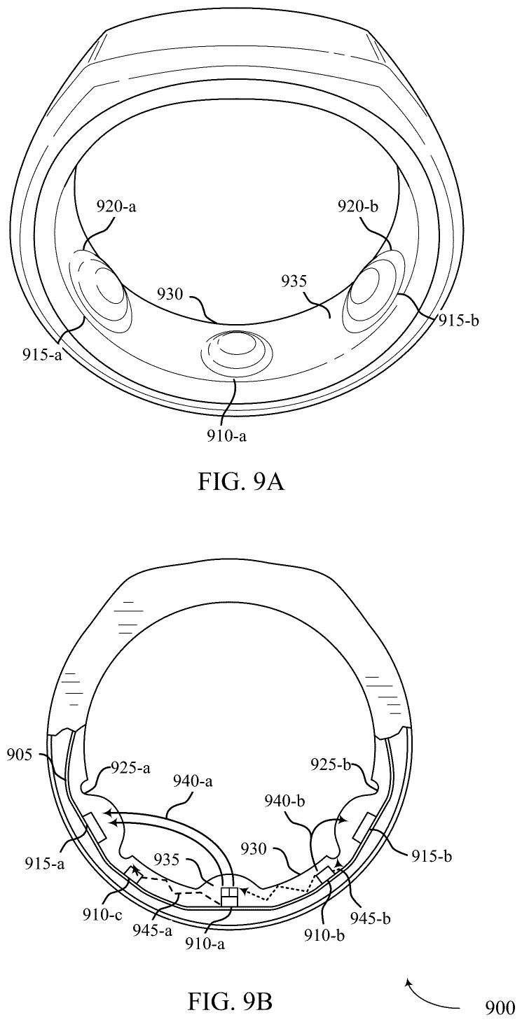

FIG. 9A illustrates an example of a perspective view of a wearable device 900 that supports optimized structures for optical measurement in accordance with aspects of the present disclosure. The wearable device 900 may implement, or be implemented by, aspects of the system 100, system 200, wearable device diagram 300, wearable device diagram 400, wearable device 500, wearable device 600, wearable device 700, wearable device 800, or a combination thereof. For example, wearable device 900 may illustrate examples of wearable devices 104 as described with reference to FIGS. 1-4. Although the wearable device 900 is illustrated as a ring in FIG. 9, aspects and components of the wearable device 900 illustrated in FIG. 9 may be implemented in any type of wearable device (e.g., a watch, a bracelet, a necklace, and the like).

As described with reference to FIGS. 5-8, the wearable device 900 may contain housing 905 which may be an example of the housing as described with reference to FIGS. 2-8. The housing 905 may contain one or more embedded sensors, such as one or more light sources 910 (e.g., LEDs) for collecting physiological measurements (e.g., physiological data) from the user. As described with reference to FIGS. 3 and 4, the light sources 910 may emit light, such that the light may be guided along to detectors 915 along the first optical path 940 and the second optical path 945.

The wearable device 900 may include the light blocking components 920. The light blocking components 920 may be an example of a light blocking component as described with reference to FIGS. 1-4. In accordance with various examples, the light blocking components 920 may be used to block stray light along the second optical path 945 from entering the detectors 915 and allow light along the first optical path 940 to enter the detectors 915, as described with reference to FIG. 4. The light blocking components 920 may include grooves 925. For example, the light blocking component 920-a may include a groove 925-a, and the light blocking component 920-b may include a groove 925-b. In some examples, one or more of the grooves 925 may extend inward from the exposed surface 930 of the layer of material 935 and towards the surface of the housing 905. The exposed surface 930 of the layer of material 935 may be configured to interface with the tissue surface. The layer of material 935 may be an example of a layer of material as described with reference to FIGS. 3-8.

The light blocking components 920 may be disposed adjacent to the detectors 915. The grooves 925 may extend at least partially around the detectors 915. In some examples, the grooves 925 may extend fully around the detectors 915 as to circumscribe the detectors 915. For examples, the grooves 925 of the light blocking components 920 may be positioned between the detectors 915 and the light sources 910 of the wearable device. In some examples, the light blocking components 920 may separate (e.g., optically isolate) the detectors 915 from the light sources 910. In some examples, grooves 925 of the light blocking components 920 may be configured to block the propagated stray light along the second optical path 945 at (e.g., near) the exposed surface 930 of the layer of material 935. For example, the grooves 925 may be sized to block the light that propagates along the second optical path 945 and within a threshold distance from the exposed surface 930 of the layer of material 935. In such examples, the light blocking components 920 may block light from entering the detectors 915 along the second optical path 945 and allow light to enter along the first optical path 940 through the tissue surface.

The grooves 925 of the light blocking components 920 may be manufactured via a molding process (e.g., an epoxy molding process). For examples, the grooves 925 may be molded from the material 935 in a molding process such that exposed surface 930 of the layer of material 935 extends inward, cutting into the inner top surface (e.g., exposed surface 930) of the layer of material 935. The grooves 925 may include a material that may include, but is not limited to, an epoxy material. In such cases, the grooves 925 of the light blocking components 920 may include a same material as the material of the layer of material 935. In some examples, the grooves 925 may include a non-transparent filler material. Without the filler material, the grooves 925 may already block stray light due to the total internal reflections from the epoxy-air interface, the elastic tissue filling the grooves 925, or both. If the grooves 925 don't include the non-transparent filler material, there may be a risk of light transmitting through walls of the grooves 925 from the light sources 910 along the second optical path 945 as well as via multiple internal reflections inside the layer of material 935. The manufacturing of the light blocking components 920 may not include additional parts for assembly because the grooves 925 may be made in a molding process similar to the process used for manufacturing if the wearable device 900 does not include the light blocking components 920.

The grooves 925 of the light blocking components 920 may include a thickness and depth that affects the amount of light that is blocked from the detectors 915 along the second optical path 945. For example, the light blocking components 920 may each have thickness of 1.05 mm and a depth of 0.50 mm from the exposed surface 930 of the layer of material 935. In some implementations, the radius of the grooves 925 may be 2.50 mm.

FIG. 9B illustrates a cross-sectional view of the wearable device 900 in accordance with aspects of the present disclosure. As previously described with respect to FIGS. 3 and 4, detector 915-a and detector 915-b may receive light emitted from the one or more light sources 910 along a first optical path 940 (e.g., through the tissue surface) and a second optical path 945 (directly from the one or more light sources 910). In such cases, the wearable device 900 may include a light blocking component 920-a and a light blocking component 920-b.

As discussed with reference to FIG. 9A, the light components 920 may include the grooves 925 surrounding the detectors 915 and extending inward from the exposed surface 930 of the layer of material 935. In some cases, the light emitted from light sources 910 may propagate along the second optical path 945 and near the exposed surface 930 of the layer of material 935 (e.g., close to the epoxy surface). For example, the light emitted from light source 910-a may propagate near the inner edges of the material 935 (e.g., near the exposed surface 930) rather than propagating through the center of the material 935. In such cases, the grooves 925 of the light blocking components 920 may block the stray light emitted from the light source 910-a (as well as light source 910-b and light source 910-c) more efficiently by extending inward from the exposed surface 930 of the layer of material 935 such that the grooves 925 block the stray light propagating along the second optical path 945 at the exposed surface 930 of the layer of material 935. For example, the grooves 925 may extend inward toward the surface of the housing 905 and at least partially through the material 935 (e.g., epoxy layer) such that the grooves 925 of the light blocking components 920 block the light traveling at the exposed surface 930 of the material 935 along the second optical path 945.

The light blocking components 920 may reduce an amount of stray light received by the detectors 915 to be 79% less stray light (e.g., along the second optical path 945) compared to a wearable device that fails to include one or more light blocking components 920. In such cases, the light blocking components 920 may allow 21% of stray light to enter the detectors 915 as compared to a wearable device that does not include a light blocking component 920. In some cases, the DC power of the PPG signal may decrease, thereby increasing a power consumption associated with the light sources 910 or other components of the wearable device 900 and decreasing a battery life of the wearable device 900. For example, the DC power consumption may include 92% of power for blood oxygen saturation and heart rate measurements using IR light (e.g., via light source 910-a) compared to a wearable device that does not include a light blocking component 920. In other examples, the DC power consumption may include 99% of power for heart rate measurements using green light (e.g., via light source 910-b and light source 910-c) compared to a wearable device that does not include a light blocking component 920.

In some cases, the use of the light blocking components 920 may increase the perfusion index less than the use of light blocking components as described with reference to FIGS. 5-8. For example, the perfusion index may increase 1.2 times for blood oxygen saturation and heart rate measurements using IR light (e.g., via light source 910-a). In other examples, the perfusion index may increase 1.3 times for heart rate measurements using green light (e.g., via light source 910-b and light source 910-c). Accordingly, the light blocking components 920 may improve the perfusion index and decrease the DC signal component.

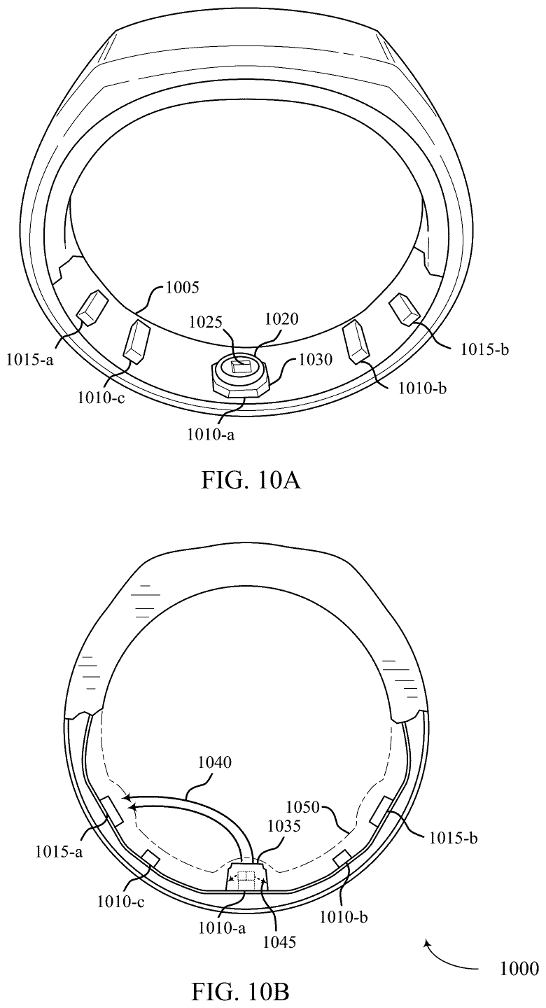

FIG. 10A illustrates an example of a perspective view of a wearable device 1000 that supports optimized structures for optical measurement in accordance with aspects of the present disclosure. The wearable device 1000 may implement, or be implemented by, aspects of the system 100, system 200, wearable device diagram 300, wearable device diagram 400, wearable device 500, wearable device 600, wearable device 700, or a combination thereof. For example, wearable device 1000 may illustrate examples of wearable devices 104 as described with reference to FIGS. 1-4. Although the wearable device 1000 is illustrated as a ring in FIG. 10, aspects and components of the wearable device 1000 illustrated in FIG. 10 may be implemented in any type of wearable device (e.g., a watch, a bracelet, a necklace, and the like).

In some examples, the wearable device 1000 may contain housing 1005 which may be an example of the housing as described with reference to FIGS. 2-9. One or more sensors may be embedded in the housing, such as one or more light sources (e.g., LEDs) for collecting physiological measurements (e.g. physiological data) from the user. As described with reference to FIGS. 3 and 4, detector 1015-a, detector 1015-b, or both may detect light emitted from one or more light sources 1010, such as light source 1010-c and light source 1010-b, respectively, and one or more additional light sources 1010 (e.g., light source 1010-a) used for physiological measurements. For example, light source 1010-a, which may be a red/IR LED, may emit light, such that the light may be guided along to detector 1015-a and detector 1015-b along the first optical path 1040 and the second optical path 1045. In some cases, the light source 1010-b and light source 1010-c, which may be green LEDs, may emit light, such that the light may be guided along to detector 1015-a and detector 1015-b along the first optical path and the second optical path.

The wearable device 1000 may include one or more light blocking components 1020. The light blocking component 1020 may be an example of a light blocking component as described with reference to FIGS. 1-4. In accordance with various examples, the light blocking component 1020 may be used to block stray light along the second optical path 1045 and allow light along the first optical path 1040 to enter the detectors 1015, as described with reference to FIG. 4.

The light blocking component 1020 may be disposed adjacent to one or more light sources 1010. For example, the light blocking component 1020 may be disposed around the light source 1010-a such that the light blocking component 1020 encompasses the light source 1010-a. In some examples, the light blocking component 1020 may be an example of a plastic molded piece at least partially surrounding the one or more light sources 1010. For example, the light blocking component 1020 may fully surround the light source 1010-a. The light blocking component 1020 may include one or more plastic materials that may include, but are not limited to, polyethylene, polypropylene, polyethylene terephthalate, or a combination thereof.

In some implementations, the light blocking component 1020 may be an example of a circular component at least partially surrounding the one or more light sources 1010. The circular component may include an opening 1025 which may include an inner cavity of the circular component. The opening 1025 of the light blocking component 1020 may be configured to direct the light from the one or more light sources 1010 (e.g., light source 1010-a) through the tissue surface along the first optical path 1040. The circular component may include a wall 1030 that blocks light from the light sources 1010 along the second optical path 1045. In such cases, the wall 1030 may block the light from entering the detectors 1015. In some examples, the light blocking component 1020 (e.g., including the wall 1030) may block the stray light traveling directly from the light sources 1010 along the second optical path 1045. The light blocking component 1020 may separate (e.g., optically isolate) the one or more light sources from the electronic circuitry of the wearable device 1000 (e.g., including the detectors 1015). In such cases, the light blocking component 1020 may limit the field of view of the light sources 1010 to be straight forward and towards the tissue surface along the first optical path 1040 rather than to the sides of the light sources 1010 (e.g., towards the detectors 1015 along the second optical path 1045).

The circular component may be an example of an injection molded piece that is placed on top of the light sources 1010 during manufacturing. For example, the light blocking component 1020 may include injection molded plastic pieces that are glued over the light sources 1010. The light blocking component 1020 may be an example of protective pieces to block the direct from the light sources 1010 from entering the detectors 1015 along the second optical path 1045. For example, the light blocking component 1020 may be glued on top of the light sources 1010 under the domes. The manufacturing of the light blocking component 1020 may include a lower cost than manufacturing of other light blocking components, but includes added complexity during assembly.

FIG. 10B illustrates a cross-sectional view of the wearable device 1000 in accordance with aspects of the present disclosure. As previously described with respect to FIGS. 3 and 4, the detectors 1015 may receive light emitted from the one or more light sources 1010 along a first optical path 1040 (e.g., through the tissue surface) and a second optical path 1045 (directly from the one or more light sources 1010). In such cases, the wearable device 1000 may include a light blocking component 1020.

The light blocking component 1020 may include one or more protrusions 1035. The one or more protrusions 1035 may be an example of the wall 1030 of the circular component. The protrusions 1035 may extend from the surface of the housing 1005. The protrusions 1035 may be configured to optically isolate the light sources 1010 from the material 1050 disposed on the surface of the housing 1005, the detectors 1015, other electrical circuitry of the wearable device 1000, or a combination thereof. In some examples, the protrusions 1035 may be configured to optically isolate the detectors 1015 from the light from the light sources 1010 along the second optical path 1045 within the layer of material 1050. The layer of material 1050 may be an example of a layer of material as described with reference to FIGS. 3-9. In some cases, the protrusions 1035 of the light blocking component 1020 may separate (e.g., isolate) the light sources 1010 from the detectors 1015 of the wearable device 1000. In such cases, the protrusions 1035 may limit the field of view of the light sources 1010 to be straight forward and towards the tissue surface (e.g., along the first optical path 1040) rather than to the sides of the light sources 1010 (e.g., along the second optical path 1045).

In some cases, the light emitted from the one or more light sources 1010 may propagate near a surface of the layer of material 1050 (e.g., close to the epoxy surface). For example, the light emitted from light source 1010-a may propagate near the outer edges of the material 1050 rather than propagating through the center of the material 1050. In such cases, the protrusions 1035 of the light blocking component 1020 may block the stray light emitted from the one or more light sources 1010 more efficiently by the protrusions 1035 extending from the surface of the housing 1005 and through the material 1050 such that the light sources 1010 are optically isolated from the detectors 1015. For example, the protrusions 1035 may protrude at least partially (e.g., most of the way) through the material 1050 (e.g., epoxy layer) such that the protrusions 1035 of the light blocking component 1020 block the light traveling at the top of the material 1050 along the second optical path 1045.

It should be noted that the methods described above describe possible implementations, and that the operations and the steps may be rearranged or otherwise modified and that other implementations are possible. Furthermore, aspects from two or more of the methods may be combined.

An apparatus is described. The apparatus may include a housing configured to house one or more sensors configured to acquire physiological data from a user, one or more light sources disposed on a surface of the housing and positioned to direct light into a tissue surface of the user, one or more detectors disposed on the surface of the housing and positioned to receive light from the one or more light sources along one or more of a plurality of optical paths, wherein a first optical path of the plurality of optical paths passes at least partially through the tissue surface and wherein a second optical path of the plurality of optical paths passes directly from one or more of the one or more light sources to one or more of the one or more detectors, and one or more light block components disposed on the surface of the housing, wherein the one or more light blocking components are configured to block light from the one or more light sources along the second optical path from entering the one or more detectors, while allowing light from the one or more light sources along the first optical path to enter the one or more detectors.

In some examples of the apparatuses, the apparatus may include a layer of material disposed on the surface of the housing and comprising optical properties that propagate the light from the one or more light sources along the second optical path within the layer of material.

In some examples of the apparatuses, the one or more light blocking components may include one or more protrusions extending from the surface of the housing, wherein the one or more protrusions may be configured to optically isolate the one or more detectors from the light from the one or more light sources along the second optical path within the layer of material.

In some examples of the apparatuses, the one or more light blocking components may include one or more grooves in the layer of material extending inward toward the surface of the housing from an exposed surface of the layer of material, wherein the one or more grooves extend at least partially around the one or more detectors and may be sized to block the light that propagates along the second optical path and within a threshold distance from the exposed surface of the layer of material.

In some examples of the apparatuses, the one or more light blocking components may include one or more circular components at least partially surrounding the one or more detectors, wherein an opening of the one or more circular components exposes the one or more detectors to the light from the one or more light sources along the first optical path, and wherein a wall of the one or more circular components blocks the light from the one or more light sources along the second optical path from entering the one or more detectors.

In some examples of the apparatuses, the one or more light blocking components may include a first wall positioned between the one or more detectors and the one or more light sources, the first wall comprising a curved portion that curves towards the one or more detectors and a second wall positioned on an opposite side of the one or more detectors from the first wall, the second wall comprising a curved portion that curves towards the one or more detectors and towards the first wall to form at least a partial dome over the one or more detectors.

In some examples of the apparatuses, the one or more light blocking components may include a first wall positioned between the one or more detectors and the one or more light sources and a second wall positioned on an opposite side of the one or more detectors from the first wall, wherein the first wall and the second wall comprise a molded and non-transparent material.

In some examples of the apparatuses, the one or more light blocking components may include a wall positioned between the one or more detectors and the one or more light sources, wherein the wall comprises an adhesive material.

In some examples of the apparatuses, the one or more light blocking components may include one or more circular components at least partially surrounding the one or more light sources, wherein an opening of the one or more circular components directs the light from the one or more light sources along the first optical path, and wherein a wall of the one or more circular components blocks the light from the one or more light sources along the second optical path from entering the one or more detectors.

In some examples of the apparatuses, the one or more light blocking components may include a material disposed on a surface of the one or more detectors, wherein the material comprises configured optical properties that allows light that enters the material at a first range of angles to pass through the material and blocks light that enters the material at a second range of angles from passing through the material, wherein the first range of angles includes at least angles of light from the first optical path, and wherein the second range of angles includes at least angles of light from the second optical path.

In some examples of the apparatuses, the housing comprises a ring-shaped housing having an inner circumference, the one or more light sources comprise one or more light-emitting diodes positioned at one or more first radial positions along the inner circumference, and the one or more detectors comprise one or more photodetectors positioned at one or more second radial positions along the inner circumference, and wherein the second optical path may be along the inner circumference from one or more of the one or more light sources to one or more of the one or more detectors.

In some examples of the apparatuses, the housing comprises a planar surface, the one or more light sources comprise one or more light-emitting diodes positioned at one or more first planar positions along the wearable device, and the one or more detectors comprise one or more photodetectors positioned at one or more second planar positions along the wearable device.

In some examples of the apparatuses, the apparatus may include a flexible printed circuit board comprising electrical circuitry for the one or more sensors.

In some examples of the apparatuses, the one or more light sources comprise one or more green light-emitting diodes, one or more red light-emitting diodes, one or more infrared light sources, or any combination thereof.

In some examples of the apparatuses, the wearable device comprises a wearable ring device.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

1. A wearable device comprising:
a housing comprising a layer of epoxy material having optical properties that facilitates light propagation through the epoxy material;
one or more light sources that are at least partially embedded within the layer of epoxy material and positioned to direct light into a tissue surface of a user;
one or more detectors that are at least partially embedded within the layer of epoxy material and positioned to receive light from the one or more light sources, wherein a first portion of the light is directed along a first optical path that passes at least partially through the tissue surface, and wherein a second portion of the light is directed along a second optical path, via the layer of epoxy material, from one or more of the one or more light sources to at least one light blocking component and towards a direction of one or more of the one or more detectors; and
one or more light blocking components comprising the at least one light blocking component that are at least partially embedded within the layer of epoxy material, wherein the one or more light blocking components are configured to block light from the one or more light sources along the second optical path from entering the one or more detectors via the layer of epoxy material while allowing light from the one or more light sources along the first optical path to enter the one or more detectors.

2. (canceled)

3. The wearable device of claim 1, wherein the one or more light blocking components comprise:
one or more protrusions that are at least partially embedded within the layer of epoxy material, wherein the one or more protrusions are configured to optically isolate the one or more detectors from the light from the one or more light sources along the second optical path via the layer of epoxy material.

4. The wearable device of claim 1, wherein the one or more light blocking components comprise:
one or more grooves in the layer of epoxy material extending inward toward a surface of the housing from an exposed surface of the layer of epoxy material, wherein the one or more grooves extend at least partially around the one or more detectors and are sized to block the light that propagates along the second optical path and within a threshold distance from the exposed surface of the layer of epoxy material.

5. The wearable device of claim 1, wherein the one or more light blocking components comprise:
one or more circular components at least partially surrounding the one or more detectors and that are at least partially embedded within the layer of epoxy material, wherein an opening of the one or more circular components exposes the one or more detectors to the light from the one or more light sources along the first optical path, and wherein a wall of the one or more circular components blocks the light from the one or more light sources along the second optical path from entering the one or more detectors via the layer of epoxy material.

6. The wearable device of claim 1, wherein the one or more light blocking components comprise:
a first wall positioned between the one or more detectors and the one or more light sources, the first wall comprising a curved portion that curves towards the one or more detectors; and
a second wall positioned on an opposite side of the one or more detectors from the first wall, the second wall comprising a curved portion that curves towards the one or more detectors and towards the first wall to form at least a partial dome over the one or more detectors.

7. The wearable device of claim 1, wherein the one or more light blocking components comprise:
a first wall positioned between the one or more detectors and the one or more light sources; and
a second wall positioned on an opposite side of the one or more detectors from the first wall, wherein the first wall and the second wall comprise a molded and non-transparent material.

8. The wearable device of claim 1, wherein the one or more light blocking components comprise:
a wall positioned between the one or more detectors and the one or more light sources, wherein the wall comprises an adhesive material.

9. The wearable device of claim 1, wherein the one or more light blocking components comprise:
one or more circular components at least partially surrounding the one or more light sources and that are at least partially embedded within the layer of epoxy material, wherein an opening of the one or more circular components directs the light from the one or more light sources along the first optical path, and wherein a wall of the one or more circular components blocks the light from the one or more light sources along the second optical path from entering the one or more detectors via the layer of epoxy material.

10. The wearable device of claim 1, wherein the one or more light blocking components comprise:
a material disposed on a surface of the one or more detectors, wherein the material comprises configured optical properties that allows light that enters the material at a first range of angles to pass through the material and blocks light that enters the material at a second range of angles from passing through the material, wherein the first range of angles includes at least angles of light from the first optical path, and wherein the second range of angles includes at least angles of light from the second optical path.

11. The wearable device of claim 1, wherein:
the housing comprises a ring-shaped housing having an inner circumference;
the one or more light sources comprise one or more light-emitting diodes positioned at one or more first radial positions along the inner circumference; and
the one or more detectors comprise one or more photodetectors positioned at one or more second radial positions along the inner circumference, and wherein the second optical path is along the inner circumference from one or more of the one or more light sources to one or more of the one or more detectors.

12. The wearable device of claim 1, wherein:
the housing comprises a planar surface;
the one or more light sources comprise one or more light-emitting diodes positioned at one or more first planar positions along the wearable device; and
the one or more detectors comprise one or more photodetectors positioned at one or more second planar positions along the wearable device.

13. The wearable device of claim 1, further comprising:
a flexible printed circuit board comprising electrical circuitry for one or more sensors.

14. The wearable device of claim 1, wherein the one or more light sources comprise one or more green light-emitting diodes, one or more red light-emitting diodes, one or more infrared light sources, or any combination thereof.

15. The wearable device of claim 1, wherein the wearable device comprises a wearable ring device.
